(19) United States
(12) Patent Application Publication
(10) Pub. No.: US 2025/0205310 A1
(43) Pub. Date: Jun. 26, 2025

(54) METHOD FOR REVERSING BODY-WIDE AGE-ASSOCIATED FUNCTIONAL CHANGES

(71) Applicant: The Chinese University of Hong Kong, Hong Kong (CN)

(72) Inventors: Ho KO, Hong Kong (CN); Junzhe HUANG, Hong Kong (CN); Andrew John KWOK, Hong Kong (CN); Chak Yan LI, Hong Kong (CN); Lek-Hin Clement CHIU, Hong Kong (CN)

(21) Appl. No.: 18/394,367

(22) Filed: Dec. 22, 2023

(51) Int. Cl. A61K 38/26; A61B 5/05; A61P 9/00; A61P 21/00; A61P 25/28; C12Q 1/6883

(52) U.S. Cl. CPC A61K 38/26; A61B 5/05; A61P 9/00; A61P 21/00; A61P 25/28; C12Q 1/6883; C12Q 2600/158

(57) ABSTRACT

Provided herein are methods and uses for preventing and treating age-associated functional changes by administering a glucagon-like peptide 1 receptor (GLP-1R) agonist to a subject in need thereof. The functional changes involve multiple organs throughout the body.

### REFERENCE TO SEQUENCE LISTING

The Sequence Listing for this application is labeled “CUHK.237.xml” which was created on Nov. 16, 2023 and is 5,961 bytes. The entire content of the sequence listing is incorporated herein by reference in its entirety.

### BACKGROUND OF THE INVENTION

Aging is a multifaceted process encompassing various intricate biological pathways (see Cohen et al., 2022; López-Otín et al., 2023). Currently, there is a scarcity of pharmaceutical interventions available for effectively preventing and treating age-associated functional decline. Inhibiting the kinase activity of mechanistic target of rapamycin (mTOR) with rapamycin has been demonstrated to improve healthspan and lifespan in diverse model organisms (see, e.g., (Kaeberlein et al., 2005; Mannick & Lamming, 2023; Vellai et al., 2003)). GLP-1 is a peptide hormone that is mainly produced by L cells in the intestine and preproglucagon neurons in the brain to regulate micronutrient balance and food intaking behaviors. GLP-1R is expressed on pancreatic beta cells. Activation of the GLP-1R on these specific cells by GLP-1 or its analogs, collectively called GLP-1R agonists (GLP-1RAs), triggers insulin secretion and safeguards these cells against apoptosis (see, e.g., (Kreiner et al., 2022; McLean et al., 2021)). Targeting this pathway has led to the development and approval of several GLP-1RAs for the treatment of type 2 diabetes mellitus (T2DM) (see, e.g., (Hinnen, 2017)). In addition to their glycemic control properties, GLP-1RAs have demonstrated remarkable benefits in reducing the cardiovascular events, preventing kidney function deterioration, and lowering the risk of stroke among diabetic patients (see, e.g., (Fan et al., 2019; Heuvelman et al., 2020; Miller et al., 2008; Mima et al., 2012)). Furthermore, GLP-1RAs also demonstrate neuroprotection benefits in preclinical models and clinical studies (see, e.g., (Athauda et al., 2017; Aviles-Olmos et al., 2014; Bertilsson et al., 2008; Y. Li et al., 2009, 2010; Perry et al., 2003)). In previous studies, we have demonstrated that in the mouse model, exenatide, one of the GLP-1RAs, ameliorates age-related cerebrovascular dysfunction and reverses aging-associated transcriptomic signatures in multiple brain cell types (Z. Li et al., 2021; Zhao et al., 2020). However, aging involves nearly all cell types and cellular processes.

Thus, there is a need for interventions for effectively preventing and treating age-associated functional decline in many cell types and cellular processes.

### BRIEF SUMMARY OF THE INVENTION

Described herein are methods for treating a subject with aging-associated functional changes. Aspects of the methods include administering a GLP-1R agonist to a subject in need thereof, such as, for example, an individual at risk of developing or suffering from aging-associated functional changes. Aging-associated functional changes include, for example, a reduction of muscle strength and endurance, a decrease in the functionality of the heart, colon, adipose tissue, circulating white blood cells, heart, spleen, lung, skeletal muscle, liver, or kidney, or a change is levels of circulating metabolites.

The subject invention pertains to methods of reversing and/or inhibiting aging-associated functional changes in a subject by targeting the GLP-1 signaling pathway using a GLP-1R agonist (GLP-1RA), such as, for example, exenatide. In certain embodiments, the anti-aging effects in the subject are accompanied with changes in transcriptomes in multiple organs and plasma metabolomes. In certain embodiments, the molecular anti-aging effects of the GLP-1RA are dependent on the hypothalamic GLP-1R.

In certain embodiments, the subject can be at risk of developing or suffering from aging-associated functional changes that can be treated with a GLP-1R agonist (GLP-1RA), such as, for example, exenatide, liraglutide, lixisenatide, albiglutide, dulaglutide, semaglutide, taspoglutide, PF-06882961 (danuglipron), OWL-833, and/or TTP-273.

In certain embodiments, the GLP-1RA treatment can reverse and/or inhibit the transcriptomic and functional changes presented in multiple cell types within the subject, including cells of the hippocampus, frontal cortex, heart muscle tissues, or skeletal muscle tissues, or circulating white blood cells (WBCs).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show the dynamic of mouse endogenous GLP-1 levels across ages in the colon, pancreas, kidney, and skeletal muscle (FIG. 1A); in the HTH, Medulla, Pons, and Spin (FIG. 1B); in the OB, CTX, HTX, BS, CRB, and Spine (FIG. 1C), and in plasma (FIG. 1D).

FIGS. 2A-2B demonstrate the impact of exenatide on the body weight (FIG. 2A) and daily food intake (FIG. 2B) of aged mice treated with GLP-1RA (exenatide) starting from 11 months of age.

FIGS. 3A-3D illustrate the forelimb grip strength (FIG. 3A), rotarod endurance (FIG. 3B), escape latency in Barnes maze (FIG. 3C), and open field locomotor activity (FIG. 3D) of aged mice after 6 months of GLP-1RA (exenatide) treatment.

FIGS. 4A-4B depict the mass of gonadal fat (FIG. 4A) in aged mice was reduced by 6 months of GLP-1RA (exenatide) treatment, while the fasting blood glucose tolerance (FIG. 4B) was not affected.

FIG. 5 shows the differentially expressed genes in aged mouse hypothalamus that were reversed after 6 months of GLP-1RA (exenatide) treatment.

FIG. 6 shows the differentially expressed genes in aged mouse adipose tissue that were reversed after 6 months of GLP-1RA (exenatide) treatment.

FIG. 7 shows the differentially expressed genes in aged mouse heart tissue that were reversed after 6 months of GLP-1RA (exenatide) treatment.

FIG. 8 shows the differentially expressed genes in aged mouse frontal cortex that were reversed after 6 months of GLP-1RA (exenatide) treatment.

FIG. 9 shows the differentially expressed genes in aged mouse colon tissue that were reversed after 6 months of GLP-1RA (exenatide) treatment.

FIG. 10 shows the differentially expressed genes in aged mouse skeletal muscle that were reversed after 6 months of GLP-1RA (exenatide) treatment.

FIG. 11 shows the proportion of categorized differentially expressed genes (DEGs) in various aged mouse tissues after 6 months of GLP-1RA (exenatide) treatment.

FIGS. 12A-12B demonstrate the impact of exenatide on the body weight (FIG. 12A) and daily food intake (FIG. 12B) of young adult mice treated with GLP-1RA (exenatide) starting from 3 months of age.

FIGS. 13A-13D illustrate the forelimb grip strength (FIG. 13A), rotarod endurance (FIG. 13B), escape latency in Barnes maze (FIG. 13C), and open field locomotor activity (FIG. 13D) of young adult mice after 6 months of GLP-1RA (exenatide) treatment.

FIGS. 14A-14B depict the mass of gonadal fat (FIG. 14A) in the young adult mice was reduced after 6 months of GLP-1RA (exenatide) treatment, while the fasting blood glucose tolerance (FIG. 14B) was not affected.

FIG. 15 shows the differentially expressed genes in aged mouse hippocampus, frontal cortex, circulating white blood cells (WBCs), heart and skeletal muscle tissues, that were reversed after 3 months of GLP-1RA (exenatide) treatment.

FIG. 16 shows the differentially expressed genes in aged mouse hippocampus, frontal cortex, circulating white blood cells (WBCs), heart and skeletal muscle tissues, that were reversed after 3 months of mTORi (rapamycin) treatment.

FIG. 17 shows the concordance of differentially expressed genes in aged mouse hippocampus, frontal cortex, circulating white blood cells (WBCs), heart and skeletal muscle tissues, that were reversed after 3 months of GLP-1RA (exenatide) and mTORi (rapamycin) treatment.

FIG. 18 shows the transcription of hypothalamic GlpIr after shRNA-based knockdown.

FIG. 19 demonstrates the dependency of the transcriptomic reversal effect of exenatide on the hypothalamic GLP-1R.

FIGS. 20A-20F show the changes of circulating metabolites after 6-month exenatide treatment (FIG. 20A), 3-month exenatide treatment (FIG. 20B), 3-month exenatide treatment with hypothalamic GLP-1R knockdown (FIG. 20C), comparison of 3-month exenatide treatments with and without hypothalamic GLP-1R knockdown (FIG. 20D), 3-month rapamycin treatment (FIG. 20E), and comparison of 3-month treatment of exenatide and rapamycin (FIG. 20F).

### BRIEF DESCRIPTION OF THE SEQUENCES

SEQ ID NO: 1:
shRNA targeting Glp1r
GCGTCAACTTTCTTATCTTCA
SEQ ID NO: 2:
a scramble sequence
CCTAAGGTTAAGTCGCCCTCG
SEQ ID NO: 3:
Glp1r primer
CAGTGGGGTACGCACTTTCT
SEQ ID NO: 4:
Glp1r primer
TAACGAACAGCAGCGGAACT
SEQ ID NO: 5:
Gapdh primer
GGCGGAGATGATGACCCTTT
SEQ ID NO: 6:
Gapdh primer
CATCTTCCAGGAGCGAGACC

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs. The term does not denote a particular age or gender. Thus, adult, young, and new-born individuals are intended to be covered as well as male and female subjects. In some embodiments, a host tissue is derived from a subject. In some embodiments, the subject is a non-human subject.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating; reducing; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 60, 70, 80, 90, 100 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 9 months, 1 year, 1.5 years, 2 years, 2.5 years, 5 years, or more than 10 years. The frequency and duration of administration of multiple doses of the compositions is such as prevent or treat endothelial dysfunction. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for endothelial dysfunction, such as, for example, electrocardiogram, angiogram, echocardiogram, flow-mediated vasodilation test, blood-based biomarkers, magnetic resonance imaging, or positron emission tomography. In some embodiments of the invention, the method comprises administration of the compounds at several times per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, the term “elderly” refers to a subject in an age group that is past middle age. Elderly refers to a specific age group, such as, for example, subjects 50 years old or older, 60 years old or older, 65 years old or older, 70 years old or older, or 75 years old or older. The elderly subject is preferably a mammal, more preferably a human, and even more preferably a human adult, as well as at least 50, 55, 60, 65, or 70 years old. More preferably, the patient is an elderly (adult) human patient who is aged or older. The elderly patient may be male or female.

### Aging-Associated Functional Changes

During the process of aging, functional changes may occur in individual or multiple organs throughout the body of a subject.

In certain embodiments, a subject that will benefit from treatment as disclosed here include individuals that are at risk of developing or suffering from aging-associated conditions, such as, for example, physical and cognitive functional declines, and/or measurable molecular changes of any of the organs throughout the subject.

In certain embodiments, aging-associated physical functional changes can comprise the loss of muscle strength and endurance and the deposition of visceral adipose tissues. Such physical functional changes can be assessed by physical tests and body composition analysis.

In certain embodiments, aging-associated functional changes can involve multiple organs, including brain, heart, lung, kidney, intestine, pancreas, skeletal muscle, adipose, liver, blood, spleen, and bone.

In certain embodiments, aging-associated functional changes can compromise molecular changes such as, for example, changes in gene expression; transcription of DNA; translation of RNA; locations of DNA, RNA or protein in cells or tissue; and/or secretion or release of proteins, DNA, RNA, mitochondria, cellular components, and/or mitochondrial components, and/or low-molecular-weight molecules (metabolites) into the body fluids. The body fluid can be cerebrospinal fluid (CSF), blood, or plasma.

Age-related changes in these measures are defined as alterations in numerical values and/or patterns obtained by the respective methods, which fall at the extrema of distributions (e.g. top or bottom 5%, 10%, 20%, 25%, 30%) for whole population or population of the same age, or exhibit changes on repeated measurement from the same subject (e.g. 5%, 10%, 15%, 20%, 25% or more) over time.

In certain embodiments, a subject that will benefit from treatment as disclosed here include individuals that are about at least 50 years old, 60 years old, 70 years old, 80 years old, 90 years old, and usually no older than 100 years old; between the ages of about 50 and 100; or about 50, about 55, about 60, about 65, about 70, about 75, about 80, about 85, about 90, about 95 or about 100 years old, and are at risk of developing or suffering from aging-associated conditions, such as, for example, physical and cognitive functional declines, and/or measurable molecular changes of any of the organs throughout the subject.

### GLP-1R Agonist

In certain embodiments, GLP-1R agonists can be used in methods of the subject invention. GLP-1R agonists are agonists of the GLP-1 receptor. GLP-1, a natural agonist of GLP-1R, has a short duration of action. Several pharmacologically optimized GLP-1R agonists have been approved or under development for the clinical treatment of diabetes mellitus, or neurodegenerative diseases, for example, Alzheimer's disease and Parkinson's disease.

The activity of a GLP-1R agonist can be determined by assays of well-studied downstream signaling and regulatory pathways linked to GLP-1R activation, such as, for example, increased cAMP production, induced phosphorylation of ERK1/2, enhanced intracellular mobilization of calcium, and recruitment of beta-arrestin-1 and beta-arrestin-2.

Some non-limiting examples of GLP-1R agonists include exenatide, liraglutide, lixisenatide, albiglutide, dulaglutide, semaglutide, taspoglutide, PF-06882961 (danuglipron), OWL-833, and TTP-273, and LY3502970 (orforglipron). In certain embodiments, the GLP-1RA can be a derivative of exenatide, liraglutide, lixisenatide, albiglutide, dulaglutide, semaglutide, taspoglutide, PF-06882961 (danuglipron), OWL-833, and TTP-273, and LY3502970 (orforglipron). In certain embodiments, the derivative of the GLP-1RA is an antibody-GLP-1RA conjugate, such as, for example, glutazumab; a peptide-GLP-1RA conjugate; a nucleotide-GLP-1RA conjugate; or a polyethylene glycol-GLP-1RA conjugate, such as, for example, NYL01.

### Dosage and Administration

In certain embodiments, the methods described herein provide a method for the treatment of aging-associated functional changes. In one embodiment, the subject can be a mammal. In another embodiment, the subject can be a human, although the invention is effective with respect to all mammals. The method can comprise administering to the subject an effective amount of a pharmaceutical composition comprising a GLP-1R agonist that reverses the transcriptomic and functional changes in the aging subject. The subject composition can further comprise one or more pharmaceutically acceptable carriers and/or excipients, and can be formulated into preparations, for example, solid, semi-solid, liquid, or gaseous forms, such as tablets, capsules, powders, granules, ointments, solutions, suppositories, injections, inhalants, and aerosols. Pharmaceutically acceptable carriers are well known in the art and include, for example, aqueous solutions, such as water or physiologically buffered saline or other solvents or vehicles, such as polyethylene glycol, Tween-20 or olive oil, or injectable organic esters. The dosage range for the agonist can depend on the potency. In certain embodiments, large amounts of an agonist can produce a desired effect, such as, for example, a reversal in the transcriptomic and functional changes in any major cell type in the subject or a reversal in structural and/or functional changes of an organ or tissue in a subject. The dosage should not be so large as to cause adverse side effects. Generally, the dosage will vary with the agents to be used. Additionally, the age, condition, and sex of the subject can be determined by one of the skill in the art and used to determine dosage. The dosage can also be adjusted by the individual physician in the event of any complications.

In one embodiment, the composition is formulated as an orally-consumable product, such as a food item, capsule, pill, or drinkable liquid. An orally deliverable health-promoting compound is any physiologically active substance delivered via initial absorption in the gastrointestinal tract or into the mucus membranes of the mouth. The composition can also be formulated as a solution that can be administered via, for example, injection, which includes intravenously, intraperitoneally, intramuscularly, intrathecally, or subcutaneously. In other embodiments, the subject composition is formulated to be administered via the skin through a patch or directly onto the skin for local or systemic effects. The compositions can also be administered sublingually, buccally, rectally, or vaginally. Furthermore, the compositions can be sprayed into the nose for absorption through the nasal membrane, nebulized, inhaled via the mouth or nose, or administered in the eye or ear.

Orally-consumable products, according to the invention, are any preparations or compositions suitable for consumption, for nutrition, for oral hygiene, or for pleasure and are products intended to be introduced into the human or animal oral cavity, to remain there for a certain period of time, and then either to be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state. The orally consumable products, according to the invention, also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally consumable product.

Carriers and/or excipients, according the subject invention, can include any and all solvents, diluents, buffers (such as neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol), and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the composition can be made into aerosol formulations so that, for example, it can be nebulized or inhaled. Suitable pharmaceutical formulations for administration in the form of aerosols or sprays are, for example, solutions, suspensions, or emulsions. Formulations for oral or nasal aerosol or inhalation administration may also be formulated with illustrative carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600, and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance is an acceptable isotonic solution, such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the adjuvant composition can be formulated for administration via topical application onto the skin, for example, as topical solutions, which include rinse, spray, drop, lotion, gel, ointment, cream, foam, powder, solid, sponge, tape, vapor, paste, tincture, or a transdermal patch. Suitable formulations of topical applications can comprise, in addition to any of the pharmaceutically active carriers, emollients, such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants, such as glycerin, propylene glycol, polyethylene glycol, sorbitol solution, and 1,2,6 hexanetriol or permeation enhancers, such as ethanol, isopropyl alcohol, or oleic acid.

Further components can be added to the compositions as are determined by the skilled artisan, for example, buffers, carriers, viscosity modifiers, preservatives, flavorings, dyes, and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

In certain embodiments, the dose will range from about 0.001 mg/kg body weight to about 1 g/kg body weight. In some embodiments, the dose will range from about 0.001 mg/kg body weight to about 0.2 g/kg body weight, from about 0.001 mg/kg body weight to about 0.1 g/kg body weight, from about 0.001 mg/kg body weight to about 20 mg/kg body weight, from about 0.001 mg/kg body weight to about 10 mg/kg body weight, from about 0.001 mg/kg body weight to about 5 mg/kg body weight, from about 0.001 mg/kg body weight to about 2 mg/kg body weight, from about 0.001 mg/kg body weight to about 1 mg/kg body weight, from about 0.001 mg/kg body weight to about 0.2 mg/kg body weight, from about 0.001 mg/kg body weight to about 0.02 mg/kg body weight, from about 0.001 mg/kg body weight to about 0.01 mg/kg body weight. Alternatively, in some embodiments the dose range is from about 0.01 g/kg body weight to about 1 g/kg body weight, from about 0.05 g/kg body weight to about 1 g/kg body weight, from about 0.1 g/kg body weight to about 1 g/kg body weight, from about 0.2 g/kg body weight to about 1 g/kg body weight, from about 0.25 g/kg body weight to about 1 g/kg body weight, from about 0.5 g/kg body weight to about 1 g/kg body weight. In one embodiment, the dose range is from about 5 μg/kg body weight to about 50 μg/kg body weight.

A GLP-1RA that reverses the aging-associated functional changes can be given multiple times a day, such as, for example, 2-times, 3-times, 4-times, 5-times, 6-times, 7-times, 8-times, 9-times, 10-times, 11-times, or 12-times per day; once a day; less than once a day; once weekly; bi-weekly; monthly; bi-monthly; quarterly; bi-yearly; yearly; or continuously in order to achieve a therapeutically effective dose. Administration of the doses used herein can be repeated for a limited period of time, such as, for example, the doses used herein can be administered daily for several weeks, months or years. Treatment duration depends on the subject's clinical progress and responsiveness to therapy. A therapeutically effective amount is an amount of a GLP-1RA that is sufficient to produce a measurable change of gene expression and/or the function of various tissues and/or organs in the subject (see “Efficacy Measurement” below). Such effective amounts can be administered in clinical trials as well as animal studies.

GLP-1RAs useful in the invention can be administered orally, intravenously, intranasally, by inhalation, intraperitoneally, intramuscularly, subcutaneously, or intracavity. In one embodiment the GLP-1RAs used herein are administered orally, or subcutaneously to a patient.

### Efficacy Measurement

In one embodiment, the efficacy of a given treatment can be determined by the improvements in functional capabilities, as measured by numbers of tests, such as, for example, grip strength, balance, memory, recall, visuospatial awareness, verbal fluency, expressive language, executive function, gait, and dual-task, multi-task. These could be reflected as improvements in the values obtained by a test, including the forelimb grip test, rotarod test, mini-mental state examination (MMSE), the Montreal Cognitive Assessment (MoCA) and its variants, the Alzheimer's Disease Assessment Scale-Cognitive Subscale (ADAS-Cog) and the Clinical Dementia Rating (CDR) scale. A clinical improvement is defined as a change in numerical values in any of the following forms: an increase in grip strength, an increase in the walking distance before losing balance, an increase in MMSE score, an increase in MoCA score, a decrease in ADAS-Cog score, or a decreased in CDR score. Improvements in capabilities can also be reflected as the lack of decline or slower decline compared age-match population in any of these scores, such as no changes in the values of grip strength, walking distance before losing balance, MMSE, MoCA, ADAS-Cog or CDR, as with ageing they are expected to continuously decline. A score satisfying any of the criteria: 26 or below on MMSE, 25 or below on MoCA, 12 or more on ADAS-Cog, 0.5 or more on CDR are considered cognitively impaired.

In another embodiment, the efficacy of a given treatment can be determined by the reversal of aging-associated structural and related functional changes, wherein the changes can be measured by an imaging method such as, for example, magnetic resonance imaging (MRI); computed tomography (CT); and ultrasonography (US). In certain embodiments, the structural changes can be measures that reflect the functions of the heart (e.g., cardiac hypertrophy, ejection fraction of the ventricles), lung (e.g., total and/or reserve volumes), kidney (e.g., volume, thickness of renal cortex, blood perfusion), pancreas (e.g., volume, digestive enzyme production capacity), skeletal muscle (muscle bulk, compositions), adipose (volume, body distribution), liver (volume), spleen (volume), and/or bone tissue (density).

In certain embodiments, structural changes of the subject can be changes in volume of an organ or tissue, i.e., 5%, 10%, 15%, 20% or more decrease, such as, for example, brain, heart, lung, kidney, intestine, pancreas, skeletal muscle, adipose, liver, blood, spleen, and/or bone tissue. Age-related decline in these measures are defined as alterations in numerical values and/or patterns obtained by the respective imaging or recording methods, that reflect changes in structure of volume, which fall at the extrema of distributions (e.g. top or bottom 5%, 10%, 20%, 25%, 30%) for whole population or population of the same age, or exhibit changes on repeated measurement from the same subject (e.g. 5%, 10%, 15%, 20%, 25% or more) over time.

In certain embodiments, functional changes of the subject can refer to measurable changes in the subject reflecting altered states of metabolism or organ functions. Functional changes of the subject can be measured by an imaging method such as, for example, spirometry, echocardiogram (e.g., to test the ejection fraction of the heart), functional magnetic resonance imaging (fMRI), magnetic resonance imaging (MRI); hyperpolarized carbon-13 (¹³C) magnetic resonance spectroscopic imaging (MRSI), ultrasonography (US), positron emission tomography (PET), or single-photon emission computerized tomography (SPECT).

In another embodiment, the efficacy of a given treatment can be determined by the reverse of aging-associated molecular changes, wherein the changes can be assessed by measuring molecules from tissues or blood fluids, such as, for example, gene expression; transcription of DNA; translation of RNA; locations of DNA, RNA or protein in cells or tissue; secretion or release of proteins, DNA, RNA, mitochondria, cellular components, mitochondrial components into blood fluids. The body fluid can be cerebrospinal fluid (CSF), blood and/or plasma.

In certain embodiments, the calculation of differentially expressed genes (DEG) with associated raw P-value, false discovery rate (FDR)-adjusted P-value, and/or magnitude of change expressed in natural log of fold change (InFC) can be calculated for each cell type. In certain embodiments, a DEG in one in which the P-value is <0.05, preferably a FDR-adjusted P-value<0.05.

In certain embodiments, the genes and subsequent transcribed mRNA and translated protein that can be assessed in the subject methods are immune response-related genes, such as, for example, C1qa, C1qb, C1qc, C4b, B2m, Tap2, H2-D1, H2-K1; synaptic modification-related genes, such as, for example, Sparcl1, Gpc6, Tgfb2, Megf10, Mertk, Chrdl1; homeostatic function-related genes, such as, for example, Kcnj10, Kenn2, Slc1a2, Slc1a3, Slc6a1, Slc6a9, Slc6a11, Slc7a10, Slc7a11, Slc16a1, Srebf1, Gja1, Gjb6, Itpr2, Grm3, Gria2, Gabbr1, Gabbr2; homeostatic-related genes in MG cells, such as, for example, Csf2r, and P2ryl3; immune activation-related genes in MG cells such as, for example, Appe, Ccl3, Ccl4, Cd52, Cst7, Fabp5, Tyrobp, Cd14, Cd33, Ifngr1, Ly86, Map4k4; immune response0inhibitaroy genes in MG cells, such as, for example, Cd300a, Il10ra, and Il10rb; calcium signaling-related genes in SMCs, such as, for example, Camk2g, Stim1, Gsn, Atp2a2, Inpp4b, Mcur1, S100a6, and Tspo; SMC contraction-related genes, such as, for example, Mylk, Itga1, Mgh11, and Sorbs1; and cell adhesion and ECM remodeling in SMCs, such as, Col1a2, Lamb1, Itga7, Jam3, Lamb2, Itgb1, and Bsg. In certain embodiments, the genes and subsequent transcribed mRNA and translated protein that can be assessed in the subject methods are those expressed in hypothalamus tissue, such as, for example, Akr1c14, Ccl28, Atp8b1, Fignl1, Aspa, Gramd3, Trim59, Gm35315, Lpar4, Calcrl, Prrx1, LOC118567992, Zfp979, Ddias, 4930447C04Rik, Zfp938, Ndufb1, Nox1, Hacd4, Slc7a11, Cntf, Cdc42ep2, Sp7, Clec2d, Zfp977, Gadl1, Zfp469, Ada, Hif3a, Zbtb16, Hr, Tekt4, Hspa1b, C2cd4a, Eps812, Ppp1r1b, Fbxw23, Cd101, Ripk4, Bdkrb2, Grin2c, Vgl12, Cacng8, Gm20346, Sf3a2, Egr4, Hspa1a, Rsph1, Lamb3, and Ppp1ccb. In certain embodiments, the genes and subsequent transcribed mRNA and translated protein that can be assessed in the subject methods are those expressed in frontal cortex tissue, such as, for example, Dynlt1b, Sdhaf4, 1110025M09Rik, 9430078G10Rik, Hmgb3, Bex3, Tmsb10, Myct1, Ccdc116, Dbp, Apcdd1, Rnf122, Map4k1, Mypn, Il17rd, Rtbdn, Gjb2, Slc6a20a, Alx3, Foxd1, Tnxb, Ntsr1, Rab7b, Edn3, Wdr62, Ppp1r3g, Irf3, Klf14, Cryab, Hsph1, Hsp90b1, Hspa5, Hspe1, Plekhg4, Galm, Pcsk1, Rps19bp1, Mt3, Rp135a, Paqr5, Smim3, Cbln4, Adi1, Anln, Sgpp2, Tm2d3, Rps23rg1, Plagl1, Rtl8b, and Sptssb. In certain embodiments, the genes and subsequent transcribed mRNA and translated protein that can be assessed in the subject methods are those expressed in adipose tissue, such as, for example, Vmn2497, adam6b, Dmrta1, Dbx2, LOC118568475, P2ry 10b, Pgap1, Slc9a7, Zbed6, Gm2808, Gm614, Npas4, St18, Gas213, Ankdd1a, Olfr111, Atp6v0d2, Gpnmb, Gm20056, Tm4sf19, Il7r, Ms4a14, Otop1, Klra10, Ryr2, Baiap212, Gkn3, Rimklb, Tceal3, Aqp5, Ttc9, Cypla1, 2810459M11Rik, Grem2, Slitrk5, Sox 10, Acox2, Rsph1, Gm42517, Atp5k, Hbb-bt, Hba-a1, Hbb-bs, Slc2a5, Slc25a1, H2-Q10, Tafa5, Tbx1, Chchd10, and Cox8b. In certain embodiments, the genes and subsequent transcribed mRNA and translated protein that can be assessed in the subject methods are those expressed in heart tissue, such as, for example, Cnksr1, Gck, Aqp8, Hspb1, Hsp90aa1, Wnt4, Col11a2, Cd16311, Neurl1a, Map3k7cl, Tpm2, My19, Tagln, Eva1c, Gmfg, Rap 1gap, Sncg, Mylk, Slc8a2, Eps812, Prag1, Capn3, Tmem150c, Kcnc1, Vipr1, 2610044015Rik8, AW551984, Zfp442, Klh132, Tfrc, Tmem35a, Kcne1, Mlf1, Tmx1, Pirt, Smim3, Fgf9, Vegfc, Tet1, Lrch2, Car8, Klh14, Gm6712, Meox2, Ifitm1, Lepr, Gm11100, Scgb1c1, Gins1, and Lhx6. In certain embodiments, the genes and subsequent transcribed mRNA and translated protein that can be assessed in the subject methods are those expressed in colon tissue, such as, for example, Gm52351, Gdf15, Chac1, Vstm21, Ms4a2, Cma2, Mcpt9, Fcer1a, Mcpt4, Cpa3, Mcpt1, Mcpt2, Calca, Pbp2, Misp3, Cnbd2, Smin22, H4c9, Atp5k, Hypk, Ndufa2, Timm13, Uqcr11, H4c8, Tmsb10, Abca8a, Acvr1c, Gm39469, Scai, Gucy1a2, Irs1, Frem2, Cntin, Gm10033, Cd36, Scd1, Pi15, Zfp979, Gm8369, Ednrb, Tnfsf10, Hhip, Fam 126b, Lrrc 19, Phlpp2, Chrm2, Kctd12b, Il18, Cyp2c68, and Hmen1. In certain embodiments, the genes and subsequent transcribed mRNA and translated protein that can be assessed in the subject methods are those expressed in skeletal muscle tissue, such as, for example, Tnfrsf12a, Cma1, Mcpt4, Dact2, Itgb7, Banp, Nos1ap, Ppp1r14b1, Catsper4, Bmp8a, Phlda1, Slc25a30, Kif26b, Tead4, Chia1, Pifo, Tekt1, Kcnab1, Otub2, Hip1r, Syt9, Pkp2, Shc2, Jchain, Pard6b, Ptx3, Sfxn2, Socs2, Abhd18, Arhgef26, Peg3, Pfkfb3, Fsbp, Mc5r, Conf, Chrdl2, Zfp503, 4930563E22Rik, Sqle, Erfe, Cdc14a, Slc7a2, Trp53i11, Bcl6b, Ptger4, Ctla2a, Tspan6, Gpx8, Elovl3, and Fgfbp1.

In certain embodiments, circulating metabolites in the subject can be assessed, such as, for example, 3-indole carboxylic acid glucuronide (HMDB0013189), 6-Hydroxy-5-methoxyindole glucuronide (HMDB0010362), Hmba (HMDB0041901), (2e,4e,6e,8e, 10e,12e,14e,16e,18e,20e,22e)-2,4,6,8,10,12,14,16,18,20,22-tetracosaundecaenal, S-3-oxodecanoyl cysteamine (HMDB0059773), (2e,4e,8z)-n-(2-hydroxy-2-methylpropyl)-2,4,8-tetradecatrienamide, 4-Oxo-1-(3-pyridyl)-1-butanone (HMDB0062406), Methyprylon (HMDB0015239), 3,4-dimethylbenzoic acid (HMDB0002237), Asarone (HMDB0031469), Brassicanal a (KEGG ID: C11048), 4-ethyl-2,6-dihydroxyphenyl hydrogen sulfate (HMDB0128030), Andrographolide (KEGG ID: C20214), N-acetyl-l-tyrosine (HMDB0000866), Indole-3-carbidol (HMDB0005785), Valerophenone (HMDB0031208), 4-tert-amylphenol (KEGG ID: C14205, HMDB0013825), Leu-val (HMDB0028942), 3-[2-(hydroxymethyl)-4-methoxyphenyl]-6-methoxy-4-oxo-3,4-dihydro-1 (2h)-quinazolinecarbaldehyde (HMDB0040442), Olodaterol, Esmolol 1 (KEGG ID: C06980; HMDB0014333), 5-hydroxyindoleacetate (KEGG ID: C05635; HMDB0000763), Indole-3-carboxilic acid-o-sulphate (HMDB0060002), or any combination thereof.

### Materials and Methods

### Animal Subjects

C57BL/6 mice were provided by the Laboratory Animal Service Center of CUHK and maintained at a controlled temperature (22-23° C.) with an alternating 12 h light/dark cycle with free access to standard mouse diet and water. The ambient humidity was maintained at <70% relative humidity.

### Active GLP-1 Measurement

Blood samples were collected into EDTA tubes through cardiac puncture. 10 μl/mL of a DPP-IV inhibitor (DPP4-M, Sigma-Aldrich, USA) was supplemented to prevent the degradation by DPP-IV. Plasma was isolated by centrifugation at 1600×g, 4° C., for 10 min. After 3 min of transcardial perfusion of ice-cold PBS, brains were rapidly removed from the crania, and the brainstem (include midbrain, pons, and medulla), hypothalamus, cerebellum, olfactory bulbs, spinal cord, and cortex were collected on ice. Tissues from other organs were also collected. All samples were snap-frozen in liquid nitrogen and stored in −80° C. until further analysis. Tissues were homogenized in a Kimble Dounce homogenizer in ice-cold PBS supplemented with 20 μl/mL of DPP-IV inhibitor and then refrozen in −80° C. for at least 24 hours. The homogenates were further thawed on ice and clarified by centrifugation at 5000×g, 4° C. for 10 min. The active GLP-1 in the supernatant was determined by the Millipore Active GLP-1 ELISA Kit (EGLP-35K, Sigma-Aldrich, USA). The capture antibody in this kit only recognizes the active forms of GLP-1, including GLP-1 (7-37) and GLP-1 (7-36 amide). Total protein in the supernatant was measured by the BCA kit (23225, Thermo Fisher Scientific, USA).

### Glp1r Knockdown

A targeting shRNA with the following sequence (5′-GCGTCAACTTTCTTATCTTCA-3′; SEQ ID NO: 1) was employed to knockdown mouse Glp1r. The shRNA was fit into the miR-30 expressing scaffold with EF1α promoter and packaged into the recombinant AAV 2/9. An AAV containing a scramble sequence (5′-CCTAAGGTTAAGTCGCCCTCG-3′: SEQ ID NO: 2) was used as control. Another AAV for expressing EGFP with the same promoter was employed to demonstrate the infecting regions and cell types. Mice were anesthetized by i.p. injection of 150 mg/kg ketamine and 10 mg/kg xylazine and positioned on a stereotaxic frame for injection. A total of one μL of 5-6×10\hat{ }9 vg (viral genome) viruses were injected into the hypothalamus. To maximize the region of infection, viruses were delivered to four sites, with coordinates as anterior/posterior (A/P)=−1.6 mm, medial/lateral (M/L)=+/−0.25 mm, dorsal/ventral (D/V)=−5.9 mm from the dura (0.2 μL per site), or A/P=−2 mm, ML=+/−0.25 mm, DV=−5.85 mm from the dura (0.3 μL per site). Mice were allowed to recover from the injection for one month before further treatment.

Quantitative PCR (qPCR) to Measure the Transcription of Glp1r

Mice were sacrificed and transcardially perfused with 20 mL ice-cold PBS. Brains were removed from the cranium and individual brain regions were isolated and preserved in the RNAlater. Total RNA was extracted by Trizol reagent and converted to cDNA by the PrimeScript RT Master Mix (RR036, Takara, USA). The qPCR reaction was prepared with TB Green Premix Ex Taq (Tli RNase H Plus) (RR420B, Takara, USA) and ran on the QuantStudio 12K Flex Real-Time PCR system (Thermo Fisher Scientific, USA). The transcription of Glp1r was determined by the following primers: forward 5′-CAGTGGGGTACGCACTTTCT-3′ (SEQ ID NO: 3) and reverse 5′-TAACGAACAGCAGCGGAACT-3′ (SEQ ID NO: 4). The transcription of Gapdh was determined by the following primers: forward 5′-GGCGGAGATGATGACCCTTT-3′ (SEQ ID NO: 5) and reverse 5′-CATCTTCCAGGAGCGAGACC-3′ (SEQ ID NO: 6).

### Treatment

For GLP-1RA treatment, exenatide (5 nmol/kg BW) was intraperitoneally (I.P.) administered (volume: 10 mL/kg bw) daily within the indicated duration. For mTORi treatment, rapamycin (8 mg/kg BW), every other day, within the indicated duration. Control mice were treated with PBS vehicle.

### Forelimb Grip Test

A grip strength meter (Model XR501, Shanghai Xin-Ruan Instruments Inc., Shanghai, China) was used to measure the forelimb grip strength. The mouse was allowed to grip the mesh lattice with its forelimbs. The peak force to pull the mouse from the grip was recorded. Five repeated measurements were performed for each mouse with a 15 min interval between two tests.

### Rotarod Test

The rotarod (Model R03-1, Xin-Ruan Instruments Inc., Shanghai, China) consists of a 3 cm diameter rod. Mice were trained to habituate the rotating rod by walking on it at a low constant speed (4 rotations per minute (R.P.M.)) for 2 min (2 min×3 times, with 15-min intervals, at the baseline test, and 2 min×1 time at the 3 months or 6 months test). Mice were then tested with the rotation of rod accelerated constantly from 4 to 40 R.P.M over 5 min. The endpoint was reached when the mice could not withstand the rotation and fell from the rod. Mice resisting falling by grasping on the rod were also treated as reaching the endpoint.

### Barnes Maze Test

The home-made Barnes maze was a white acrylic round disk, 90 cm in diameter, with 20 holes (5-cm diameter) spaced equally located in a circumference 5 cm from the outer edge. 19 of the 20 holes were blocked while the remaining one provided access to an escape box. Visual cues were placed around the maze in the testing room. On the first day, mice were trained to identify the escape box. Mice were covered by a cardboard box in the center of the maze. Five seconds later, an aversive sound noise was initiated from a nearby device (900 Hz, 80 db), and the box was removed to permit the exploration and escape. Mice failed to find the escape box in 3 min were guided to it by a glass cylinder. One hour later, mice were tested once again by the same procedure. From day two to four, mice were tested the same way two times with 1-hour intervals. Entering the escape box or spending at least five seconds exploring the escape was defined as an escape.

### Open Field Test

The open field arena measured 58 cm×58 cm, with a wall height of 30 cm. Individual mice were allowed to explore freely in the arena for min. Video recording was done by a web-camera and analyzed by the MouseActivity open-source code, which was obtained from GitHub-HanLab-OSU/MouseActivity, and implemented in MATLAB R2021a.

### Tissue Collection and RNA Extraction

Mice were treated with the last dose of exenatide in the morning and fasted for 6 hours before termination. Mice were euthanized by isoflurane vapor, with blood collection through cardiac puncture, and then perfused with ice-cold PBS (15-20 ml) transcardially. Blood samples were lysed in an ice-cold red blood cell lysis buffer for 20 min. The white blood cells were separated via centrifugation at 500×g for 10 min. Other tissues collected included the frontal cortex, hippocampus, hypothalamus, pancreas, liver, kidney, spleen, gonadal adipose, heart (left ventricle), skeletal muscle (quadriceps femoris), colon (proximal), and lung. All tissues were preserved in RNAlater for further RNA extraction. Total RNA of the aged mice's frontal cortex, hippocampus, hypothalamus, WBCs, kidney, and pancreas was extracted by the RNAqueous kit (AM1912, Thermo Fisher Scientific, US), while total RNA of the other tissues was extracted by the Trizol reagent (15596018, Thermo Fisher Scientific, US).

### Bulk RNA-Sequencing Analysis

RNA samples were sent to Novogene (Tianjin, China) for RNA-sequencing. RNA concentration, purity and integrity were checked by Novogene. The samples passed these quality checks were subjected to library preparation. mRNAs were enriched by polyA selection, and the whole transcriptome was sequenced on Illumina Hiseq-PE150 platform in a paired-end, non-stranded protocol. Quality control of raw reads data, alignment of reads and quantification of transcripts were done on the Linux system (Ubuntu 20.04.4 LTS). QC of the raw reads and alignment files were conducted using the FastQC v.0.11.9 and MultiQC v.1.13a packages. Reads were aligned against the mouse genome reference GRCm39 by using STAR v.2.7.10b with default parameters. Transcript quantification was done by HTSeq v.2.0.2 with default settings except ‘mode’ was set to ‘intersection-strict’ and ‘nonunique’ was set to ‘fraction’ for ambiguous reads. Batch effects were identified and corrected by the removeBatchEffect function in the Limma v.3.52.4 package. Customized R scripts (R v.4.2.2) running on RStudio 2023.3.0.386 were used in the downstream analysis. Differential gene expression (DGE) analysis was performed on DESeq2 v.1.36.0. To remove low-expression genes, a pseudocount of 1 was added and genes with more than 5 counts in at least n samples were kept for further analysis, where n was the minimum of the sizes of the experimental groups involved (n=5 for WBCs, n=7 for the colon, and n=8 for the other tissues). Counts normalization and variance-stabilizing transformation were conducted by the built-in algorithm in DESeq2. The apeglm v.1.18.0 package was used to obtain log₂ fold changes in each comparison. The default Benjamini-Hochberg method was used to calculate the corresponding P-values adjusted for false discovery rate (FDR).

### Plasma Metabolome Profiling

Peripheral blood samples were collected in EDTA tubes and processed immediately. Plasma was isolated by centrifuging the blood sample at 1,600×g under 4° C. for 15 minutes and stored at −80° C. until further assays. To extract the metabolites, 100 μl of plasma were combined with 700 μl of extractant containing internal standard (methanol:acetonitrile:water=4:2:1, v/v/v), shaken for 1 min and placed at −20° C. for 2 hours. The samples were then spun at 25,000×g under 4° C. for 15 min. The supernatant was transferred to a new EP tube and dried with the solvent. The pellet was reconstituted in 180 μL of methanol:water (1:1 v/v) and spun again at 25,000×g under 4° C. for 15 min. The resulting supernatant was subjected to LC-MS/MS analysis (Waters UPLC I-Class Plus (Waters, USA) tandem Q-Exactive high resolution mass spectrometer (Thermo Fisher Scientific, USA)). A BEH C18 column was employed to identify the metabolites. This column preferentially detects non-polar and medium-polar small molecules. The off-line data of mass spectrometry were imported into Compound Discover™ 3.3 software (Thermo Fisher Scientific, USA) and analyzed in combination with the BGI metabolome, mzCloud, and ChemSpider online databases. A data matrix containing information such as metabolite identification and peak area was obtained for further analysis. The peak areas were normalized by Probabilistic Quotient Normalization (PQN). Batch effects were corrected by quality control-based robust LOESS signal correction, if necessary. Further analysis and statistical comparisons were conducted with custom-written codes in R and/or Python.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Level of Active Hypothalamic GLP-1 Decreases as Mice Age

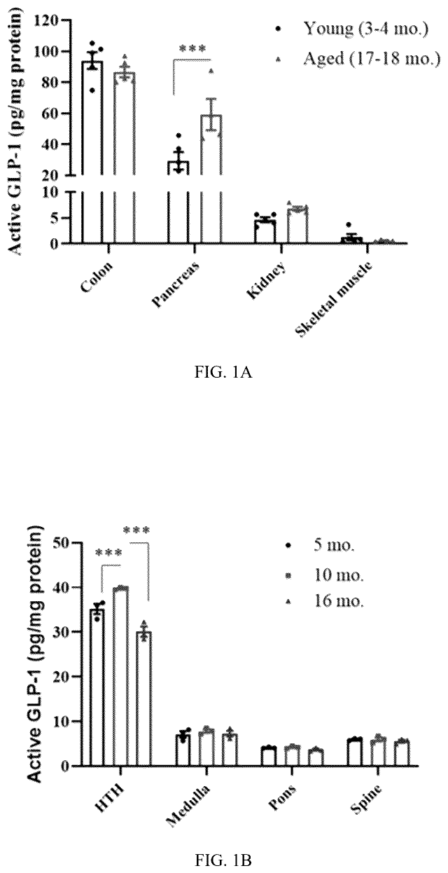

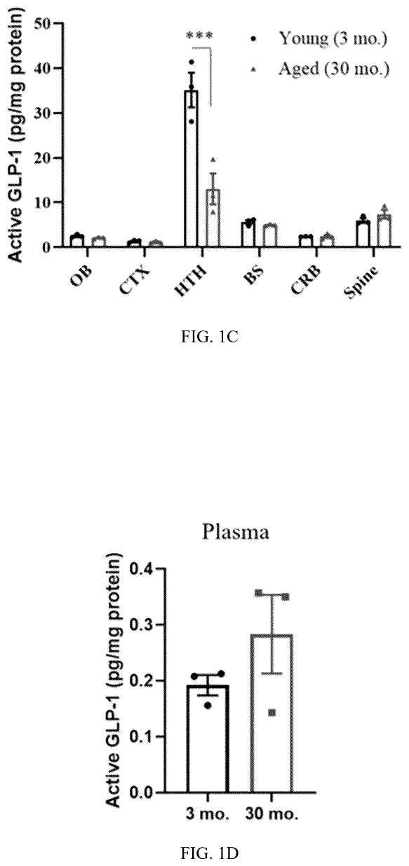

FIGS. 1A-1D demonstrates the levels of active GLP-1 in various organs across different ages. In line with that the L-cells and the α-cells are the main sources to produce GLP-1 in the periphery, the highest levels of active GLP-1 were detected in the colon and the pancreas. The colon GLP-1 in the aged mice was slightly lower than that in the young mice (mean 86.56 vs 93.94, Sidak's multiple comparisons test, adjusted P=0.6447). Conversely, pancreatic GLP-1 in the aged mice was significantly higher than that in the young mice (man 59.12 vs 29.35, Sidak's multiple comparisons test, adjusted P=0.0002). Active GLP-1 in the aged kidney also increased slightly but without a statistically significant difference. Active GLP-1 in the skeletal muscle was low compared to the other tissues with no significant difference between the groups. The central GLP-1 levels in a cohort of 3-, 10- and 16-months old mice were measured (FIG. 1B). The highest level of active GLP-1 was detected in the hypothalamus. The results showed a bell-shape of the hypothalamic GLP-1 levels, which went up from 3 months to 10 months, and then down from 10 months to 15 months of ages (means were 35.16, 39.74, and 30.09 respectively). Comparisons between any two ages were significant (two-way ANOVA, Sidak's multiple comparisons test, adjusted P all <0.0001). There were no age-associated differences in active GLP-1 levels in the other brain regions tested. The central GLP-1 levels were further measured in another cohort of mice that were 3 or 30 months old (FIG. 1C). Tissues were collected from the olfactory bulb (OB), cortex (CTX), hypothalamus (HTH), brain stem (BS), cerebellum (CRB), and spinal cord (spine). The hypothalamic GLP-1 dramatically decreased in aged mice (man 13.05 vs 35.11, two-way ANOVA, Sidak's multiple comparisons test, adjusted P<0.0001). The plasma GLP-1 was very low and exhibited no significant difference between the two groups (FIG. 1D).

### Example 2—Effects of 6-Month Exenatide Treatment on the Body Weight and Food Intake of Aged Mice

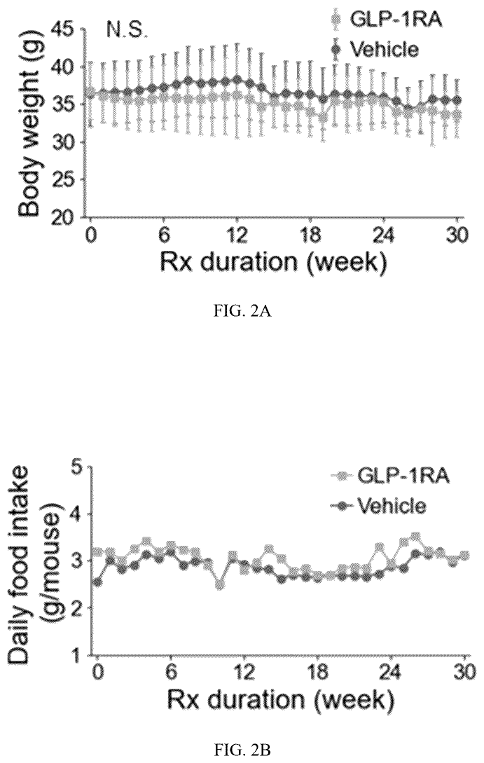

Body weight and food intake were monitored during the treatment period (FIGS. 2A-2B). Two-way RM ANOVA analysis only showed a significant time effect on the BW (time effect P=0.0007; treatment effect P=0.4368; interaction P=0.1508; post-hoc Sidak's tests showed no significant differences at any individual time points). Daily food intake was not significantly affected by the treatment.

### Example 3—6 Months of Exenatide Treatment Improved the Exercise Capacities of Aged Mice

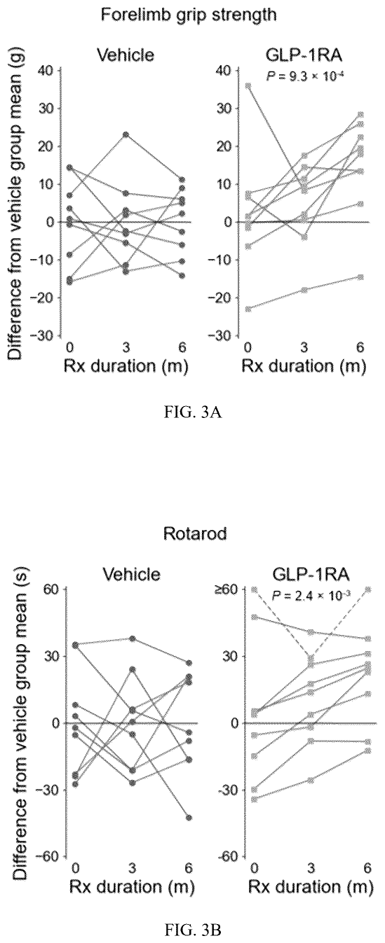

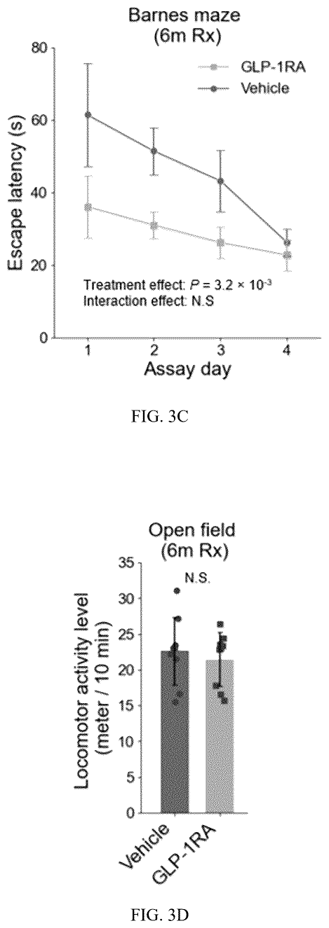

The exercise capacities were measured by the forelimb grip strength, accelerated rotarod latency, and open field (FIGS. 3A, 3B, and 3D). Mice in the treatment group showed significant improvements in forelimb grip strength and rotarod performance. No significant effects were observed in the open field test.

### Example 4—6 Months of Exenatide Treatment Improved the Spatial Reference Memory of Aged Mice

The Barnes maze task relies on rodent's intrinsic preference for dark, enclosed spaces over open areas (Harrison et al., Learning & Memory. 13:809 (2006)). The task requires the tested mice to learn the position of the escape hole by using some spatial reference. Exenatide treatment significantly improved the performance (FIG. 3C, treatment effect: P=0.0032).

### Example 5—6 Months of Exenatide Treatment Reduced the Mass of Gonadal Fat Tissue in Aged Mice

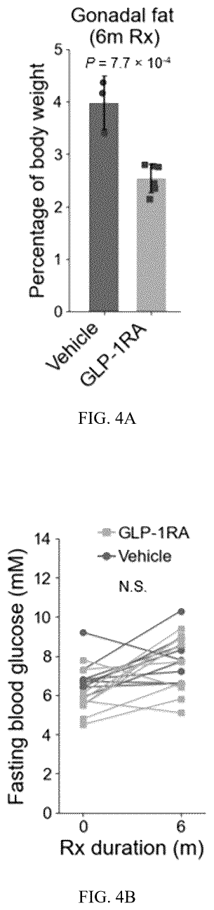

The mass of the gonadal fat tissues collected was measured when the mice were euthanized at the end point. Exenatide treatment significantly reduced the gonadal fat mass to body weight ratio (FIG. 4A, P=7.7×10−4). By contrast, the treatment did not alter the fasting blood glucose (FIG. 4B).

### Example 6—6 Months of Exenatide Reversed Body-Wide Aging-Associated Transcriptomic Changes in Aged Mice

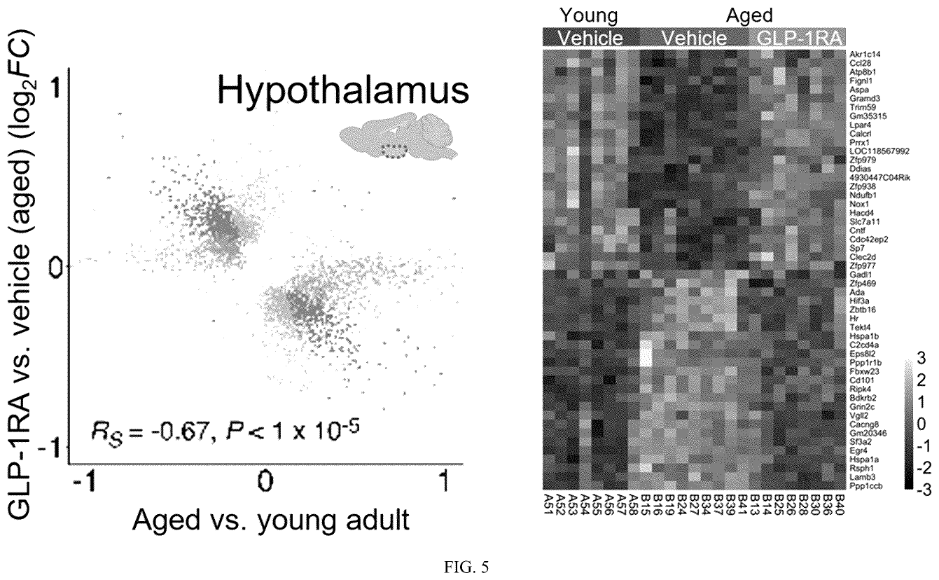

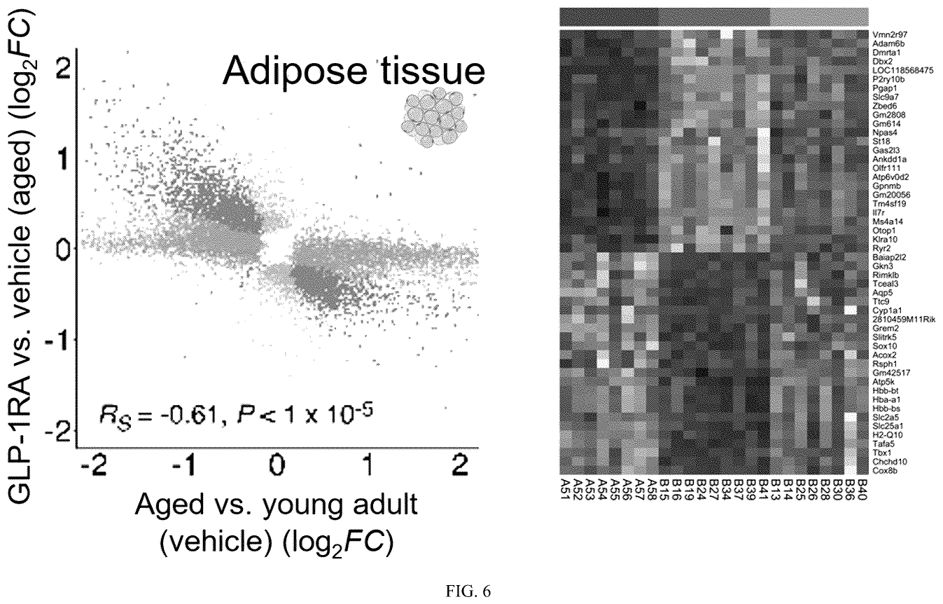

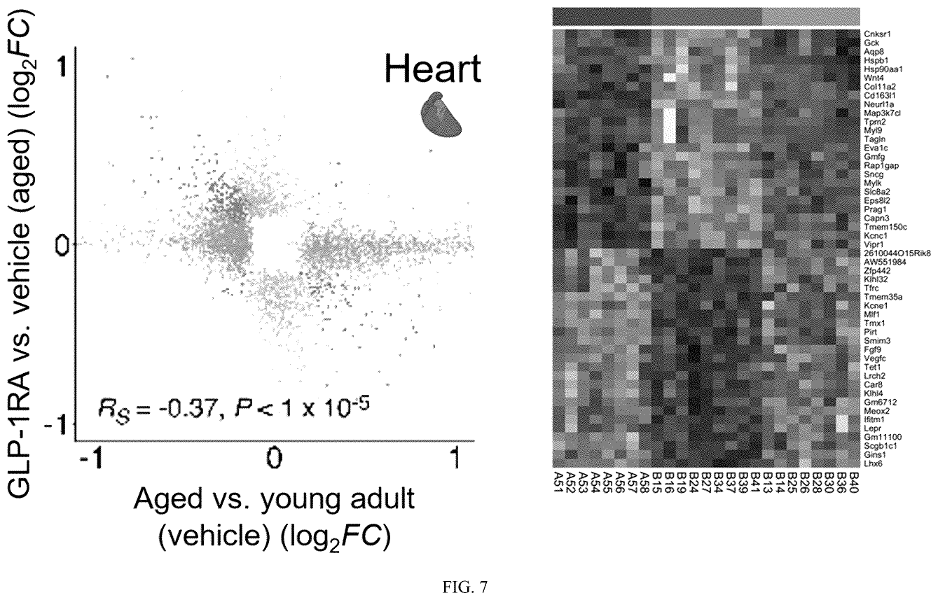

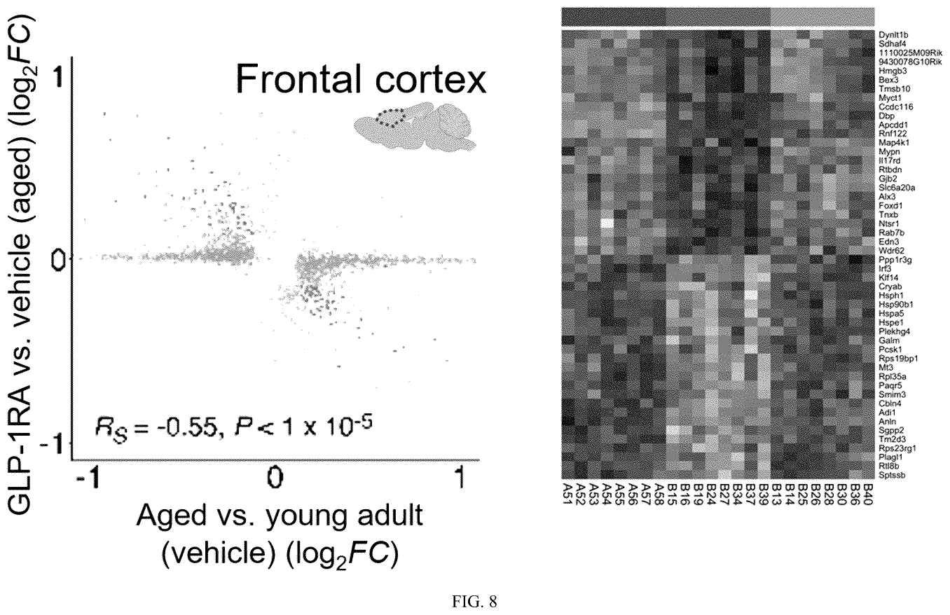

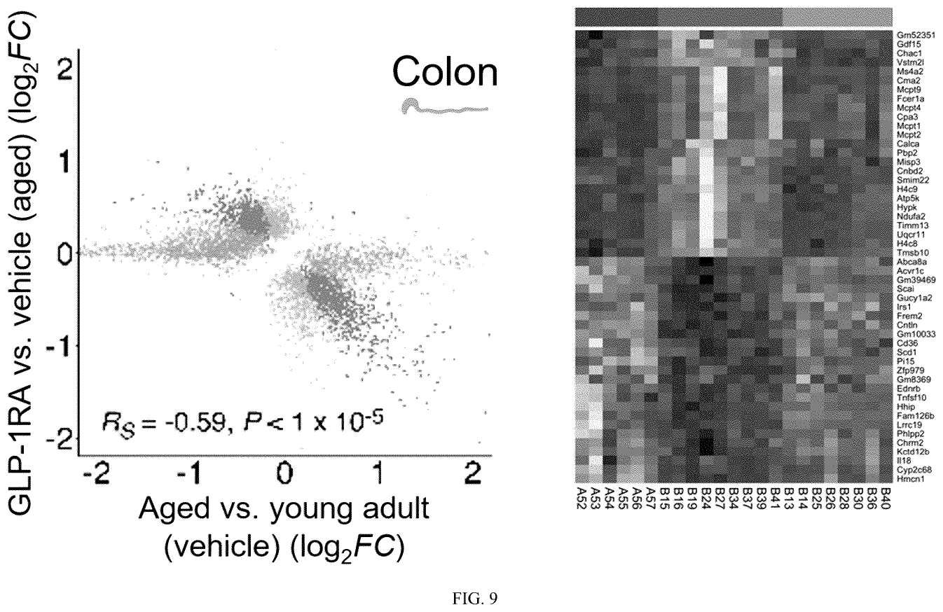

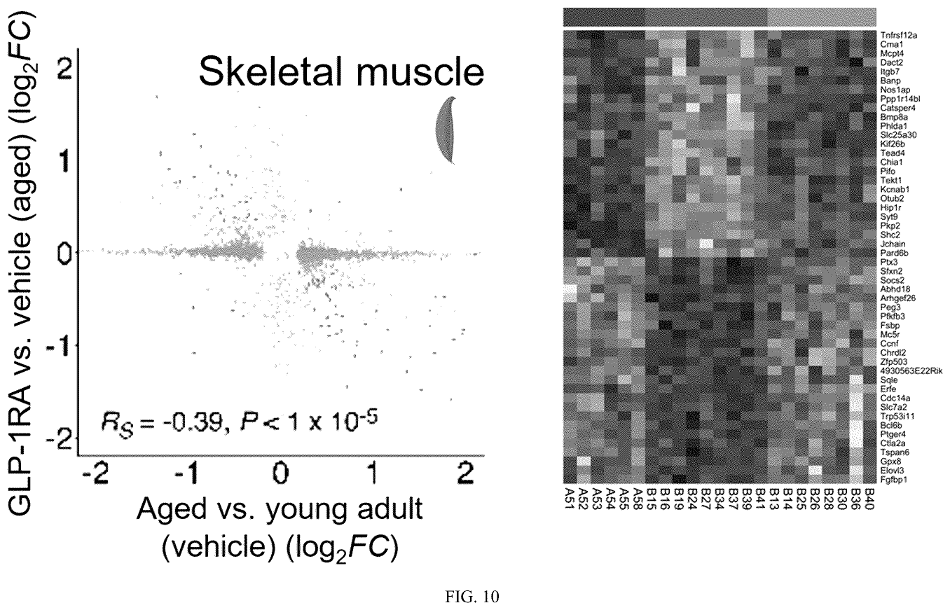

Two comparisons for the gene expression levels were performed, one between the aged-vehicle group and the young group, the other between the aged-exenatide group and the aged-vehicle group. The identified DEGs (differentially expressed genes) exhibited a significant negative correlation between these two comparisons in tissues of organs including hypothalamus, adipose, heart, frontal cortex, colon, and skeletal muscle (FIGS. 5-10). Examples of top aging-associated DEGs that were reversed by exenatide are shown in the heatmap.

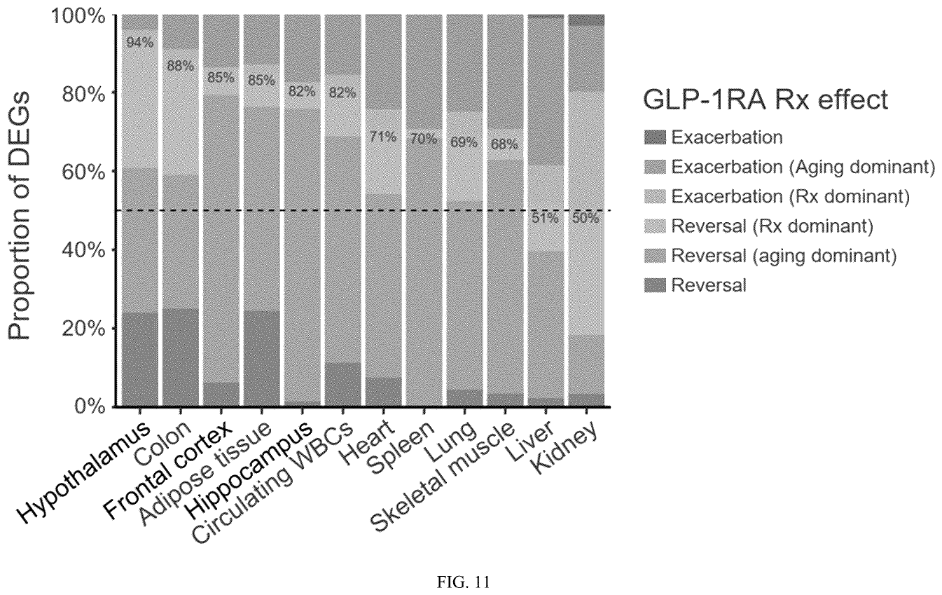

The DEGs were sorted into six categories, according to the change directions and effect size. Category 1: Exacerbation, which means the expression of the genes in this class was significantly increased or reduced in aged-vehicle group compared to young group, and exenatide treatment led to a change at the same direction (exacerbated) with statistical significance. Category 2: Exacerbation (aging dominant), which means the expression of the genes in this class was significantly increased or reduced in aged-vehicle group compared to young group, exenatide treatment led to the same direction of change but without statistical significance. Category 3: Exacerbation (Rx dominant), which means the expression of the genes in this class was increased or reduced in aged-vehicle group compared to young group without significant differences, but exenatide treatment exacerbated the change with statistical significance. Category 4: Reversal, which means the expression of the genes in this class was significantly increased or reduced in aged-vehicle group compared to young group, while exenatide treatment led to a change at the opposite direction (reversal) with statistical significance. Category 5: Reversal (aging dominant), which means the expression of the genes in this class was significantly increased or reduced in aged vehicle group compared to young group, and this change was reversed by exenatide without statistical significance. Category 6: Reversal (Rx dominant), which means the expression of the genes in this class was increased or reduced in aged vehicle group compared to young group without statistical significance, but this change was significantly reversed by exenatide. FIG. 11 demonstrates in most of the tissues, the majority of DEGs belong to the reversal categories (including both aging dominant and Rx dominant).

### Example 7—6 Months Exenatide Treatment has Less Effects on Young Adult Mice

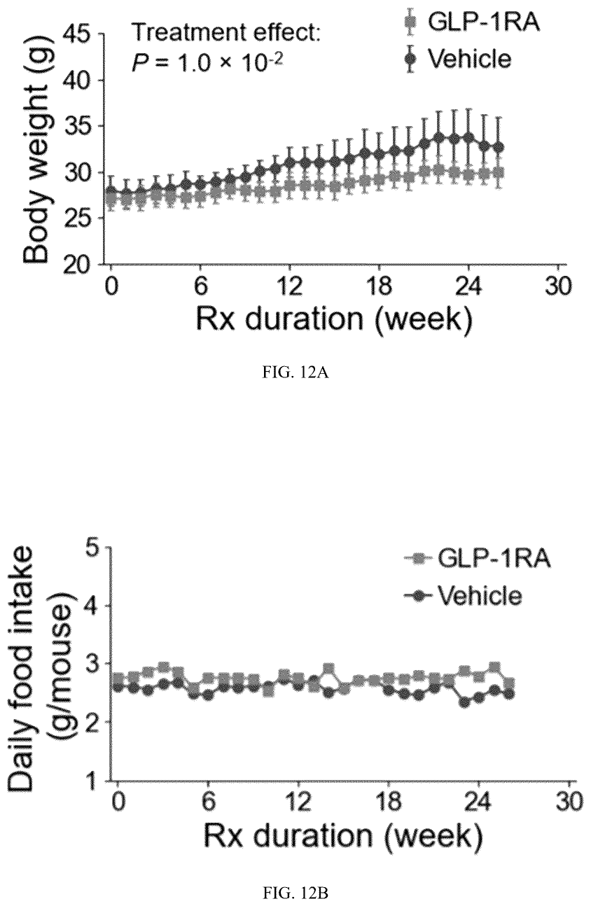

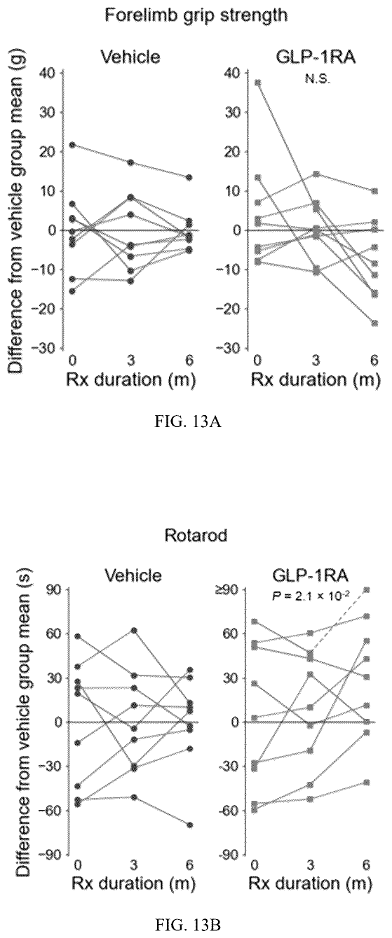

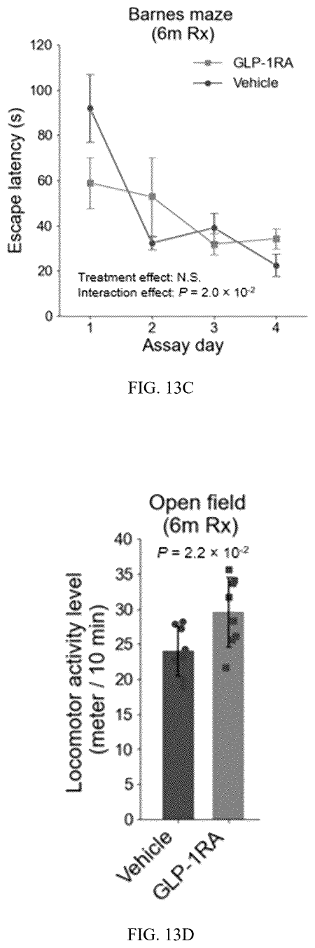

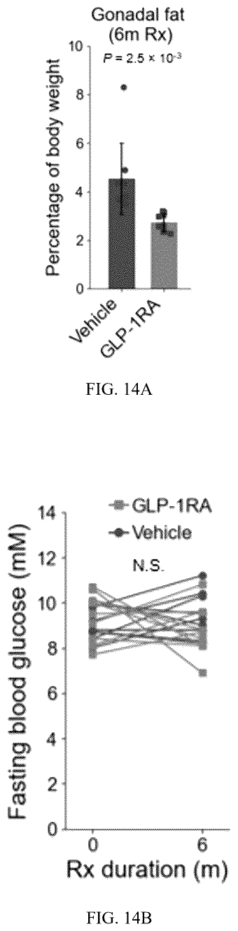

Young adult mice treated with exenatide had a significantly lower body weight, around 12% difference at the maximum (FIGS. 12A-12B, two-way RM ANOVA, treatment effect P=0.0121). Daily food intake was not significantly affected by the treatment (FIGS. 12A-12B). Exenatide treatment had no effect on the forelimb grip strength (FIG. 13A), while it improved the performance of mice on the rotarod (FIG. 13B) and open field (FIG. 13D). Barnes maze showed no difference regarding the treatment effect, while there was a significant difference in the interaction effect between treatment and assay days (FIG. 13C). The treatment led to a significant reduction in gonadal fat mass (FIG. 14A), while having little effect on the fasting blood glucose (FIG. 14B).

### Example 8—3 Months of Exenatide Treatment Reversed Body-Wide Aging-Associated Transcriptomic Changes in Aged Mice

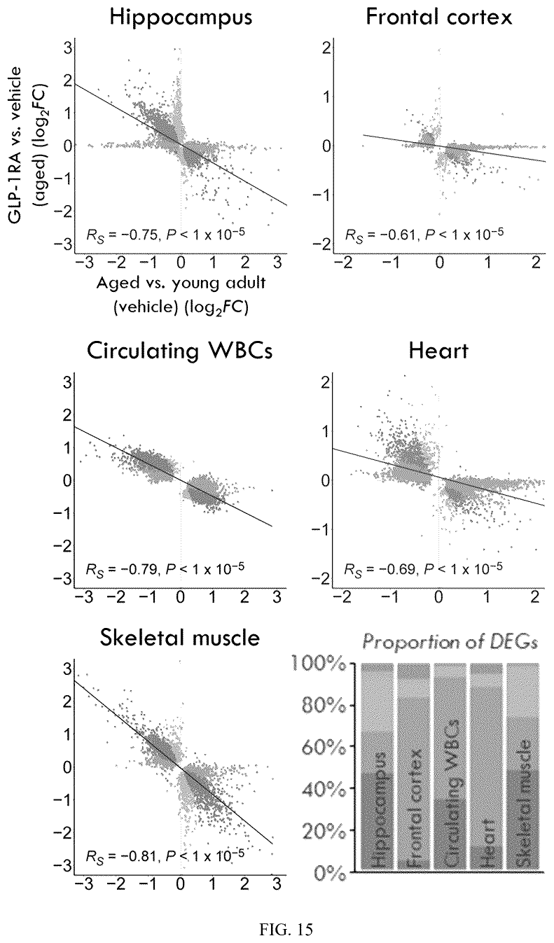

We treated another cohort of aged mice with exenatide, which began at 18 months of age and lasted for 3 months. We profiled the transcriptomes of hippocampus, frontal cortex, circulating white blood cells (WBCs), heart, and skeletal muscle. Even with this short term treatment regimen, exenatide exhibited notable and statistically significant anti-aging effects, as depicted in FIG. 15.

### Example 9—the Transcriptomic Anti-Aging Effects of Exenatide in Several Organs are Comparable with Rapamycin

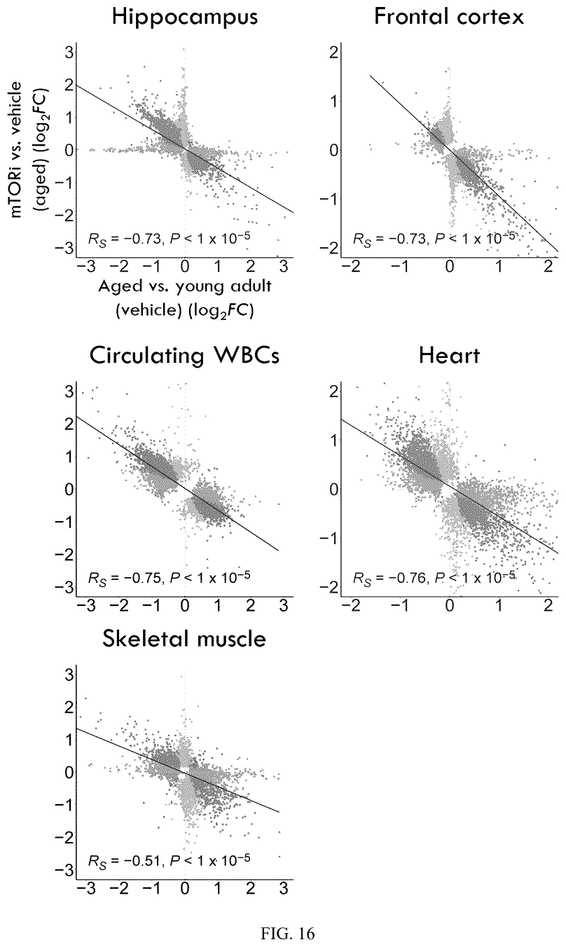

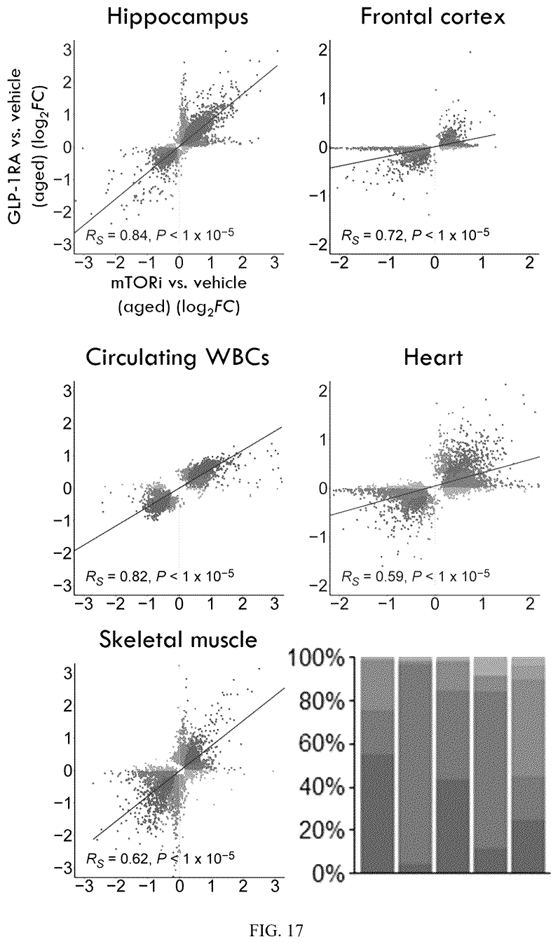

In conjunction with the study described in Example 8, we treated an additional group of aged mice with mTOR inhibitor (mTORi), rapamycin, a potential anti-aging agent that has been well-studied. Rapamycin exhibited remarkable and statistically significant anti-aging effects, as evidenced by the transcriptomes of the hippocampus, frontal cortex, circulating WBCs, heart, and skeletal muscle (FIG. 16). Furthermore, we identified the DEGs that were common to both exenatide and rapamycin treatments. These DEGs displayed a high level of agreement in terms of the direction and magnitude of their changes, indicating a comparable and correlated treatment effect between exenatide and rapamycin (FIG. 17).

### Example 10—the Transcriptomic Anti-Aging Effects of Exenatide is Sensitive to Hypothalamic GLP-1R Knockdown

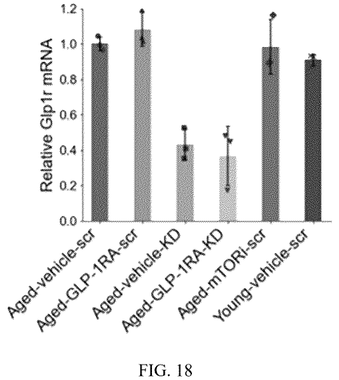

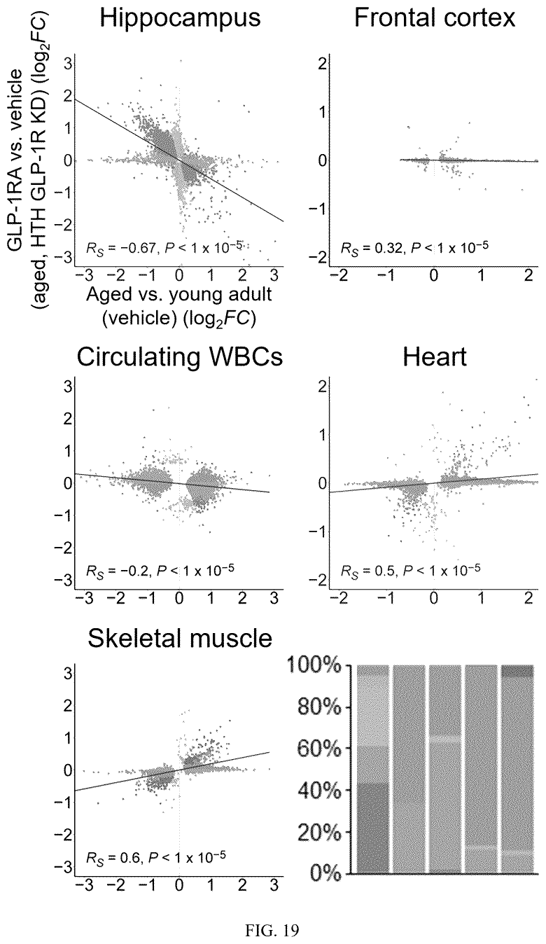

In conjunction with the study described in Example 8, we knocked down hypothalamic GLP-1R in an additional group of aged mice. The knockdown efficiency was shown in FIG. 18. We investigated the impact of hypothalamic GLP-1R on the treatment effects by comparing the transcriptomic changes on these knockdown mice. As depicted in FIG. 19, the treatment effects exhibited a significant reduction in the frontal cortex, circulating WBCs, heart, and skeletal muscle, following the knockdown of hypothalamic GLP-1R. However, the treatment effects on the hippocampus were relatively less affected by the GLP-1R knockdown.

### Example 11—Treatment Effects on the Circulating Metabolites

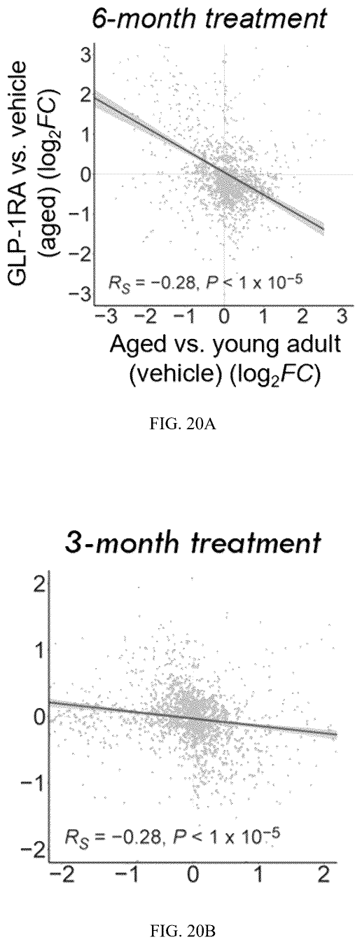

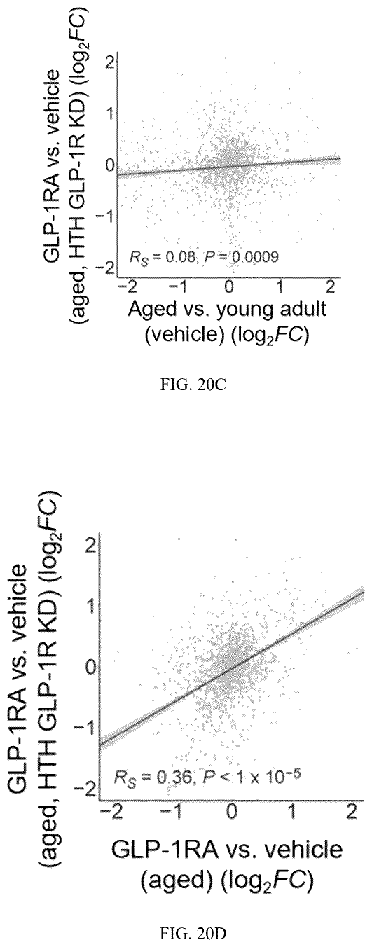

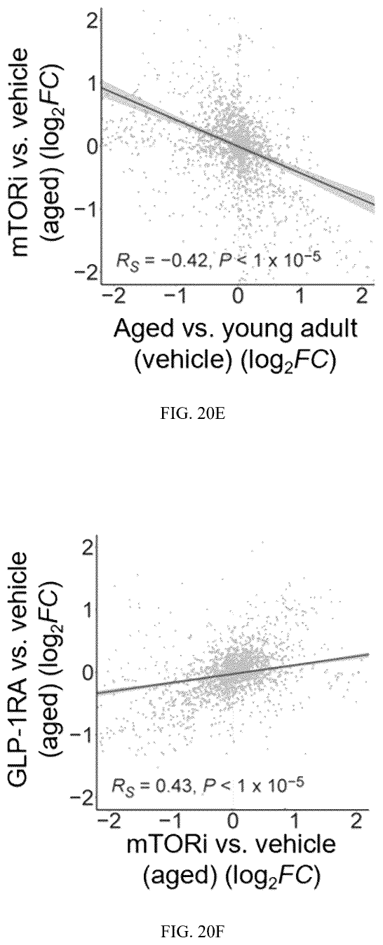

Following 6 months of exenatide treatment, a dramatic reversal of the circulating metabolites was observed (FIG. 20A). However, with a shorter treatment duration of 3 months, the reversal effect was less pronounced (FIG. 20B). The reversal effect was partially dampened when the hypothalamic GLP-1R was knocked down (FIGS. 20C-20D). In addition to exenatide, aged mice treated with rapamycin also exhibited a strong reversal in the circulating metabolites (FIG. 20E). The changes in circulating metabolites induced by exenatide treatment were positively correlated with those by rapamycin treatment (FIG. 20F).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

### Embodiment 1

A method for decreasing an aging-associated functional change in a subject, the method comprising administering to the subject an effective amount of a glucagon-like peptide 1 receptor agonist (GLP-1RA); wherein the GLP-1RA is selected from the group consisting of exenatide, liraglutide, lixisenatide, albiglutide, dulaglutide, semaglutide, taspoglutide, PF-06882961, OWL-833, TTP-273, LY3502970, a derivative of exenatide, a derivative of liraglutide, a derivative of lixisenatide, a derivative of albiglutide, a derivative of dulaglutide, a derivative of semaglutide, a derivative of taspoglutide, a derivative of PF-06882961, a derivative of OWL-833, a derivative of TTP-273, and a derivative of LY3502970.

### Embodiment 2

The method of embodiment 1, wherein the subject is a mammal.

### Embodiment 3

The method of embodiment 2, wherein the mammal is a primate.

### Embodiment 4

The method of embodiment 3, wherein the primate is a human.

### Embodiment 5

The method of embodiment 1, wherein the subject is 50 years or older.

### Embodiment 6

The method of embodiment 1, wherein the aging-associated functional change is a reduction of muscle strength, balance, muscle endurance, or any combination thereof.

### Embodiment 7

The method of embodiment 1, wherein the aging-associated functional change is a decrease in heart ejection fraction.

### Embodiment 8

The method of embodiment 1, wherein the aging-associated function impairment is cognitive impairment.

### Embodiment 9

The method of embodiment 1, wherein the aging-associated functional change is a measurable change in colon activity, adipose tissue amount, circulating white blood cell amount, spleen activity, lung airflow, skeletal muscle activity, heart activity, liver activity, kidney activity, or any combination thereof.

### Embodiment 10

The method according to embodiment 9, wherein the measurable change is measured by a functional imaging or recording method.

### Embodiment 11

The method according to embodiment 10, wherein the functional imaging or recording method is spirometry, echocardiogram, functional magnetic resonance imaging (fMRI), magnetic resonance imaging (MRI), hyperpolarized carbon-13 (¹³C) magnetic resonance spectroscopic imaging (MRSI), ultrasonography (US), positron emission tomography (PET), single-photon emission computerized tomography (SPECT), electroencephalography (EEG), magnetoencephalography (MEG), functional near-infrared spectroscopy (fNIRS), intracortical electrode recording, or deep brain electrode recording.

### Embodiment 12

The method of claim 1, wherein the derivative of exenatide is an exenatide-antibody conjugate, an exenatide-peptide conjugate, an exenatide-nucleotide conjugate, or an exenatide-polyethylene glycol conjugate;
the derivative of liraglutide is a liraglutide-antibody conjugate, a liraglutide-peptide conjugate, a liraglutide-nucleotide conjugate, or a liraglutide-polyethylene glycol conjugate;
the derivative of lixisenatide is a lixisenatide-antibody conjugate, a lixisenatide-peptide conjugate, a lixisenatide-nucleotide conjugate, or a lixisenatide-polyethylene glycol conjugate;
the derivative of albiglutide is an albiglutide-antibody conjugate, an albiglutide-peptide conjugate, an albiglutide-nucleotide conjugate, or an albiglutide-polyethylene glycol conjugate;
the derivative of dulaglutide is a dulaglutide-antibody conjugate, a dulaglutide-peptide conjugate, a dulaglutide-nucleotide conjugate, or a dulaglutide-polyethylene glycol conjugate;
the derivative of semaglutide is a semaglutide-antibody conjugate, a semaglutide-peptide conjugate, a semaglutide-nucleotide conjugate, or a semaglutide-polyethylene glycol conjugate;
the derivative of taspoglutide is a taspoglutide-antibody conjugate, a taspoglutide-peptide conjugate, a taspoglutide-nucleotide conjugate, or a taspoglutide-polyethylene glycol conjugate;
the derivative of PF-06882961 is a PF-06882961-antibody conjugate, a PF-06882961-peptide conjugate, a PF-06882961-nucleotide conjugate, or a PF-06882961-polyethylene glycol conjugate;
the derivative of OWL-833 is an OWL-833-antibody conjugate, an OWL-833-peptide conjugate, an OWL-833-nucleotide conjugate, or an OWL-833-polyethylene glycol conjugate;
the derivative of TTP-273 is a TTP-273-antibody conjugate, a TTP-273-peptide conjugate, a TTP-273-nucleotide conjugate, or a TTP-273-polyethylene glycol conjugate; or
the derivative of LY3502970 is a LY3502970-antibody conjugate, a LY3502970-peptide conjugate, a LY3502970-nucleotide conjugate, or a LY3502970-polyethylene glycol conjugate.

### Embodiment 13

The method of embodiment 1, wherein the aging-associated functional change is a change in an expression of at least one gene in the subject.

### Embodiment 14

The method of embodiment 1, wherein the aging-associated functional change is a change in a level of at least one circulating metabolite in the subject.

### Embodiment 15

The method of embodiment 13, wherein the at least one gene is C1qa, C1qb, C1qc, C4b, B2m, Tap2, H2-D1, H2-K1, Sparcl1, Gpc6, Tgfb2, Megf10, Mertk, Chrdl1, Kcnj10, Kcnn2, Slc1a2, Slc1a3, Slc6a1, Slc6a9, Slc6a11, Slc7a10, Slc7a11, Slc16a1, Srebf1, Gja1, Gjb6, Itpr2, Grm3, Gria2, Gabbr1, Gabbr2, Csf2r, P2ryl3, Appc, Ccl3, Ccl4, Cd52, Cst7, Fabp5, Tyrobp, Cd14, Cd33, Ifngr1, Ly86, Map4k4, Cd300a, Il1Ora, I110rb, Camk2g, Stim1, Gsn, Atp2a2, Inpp4b, Mcur1, S100a6, Tspo, Mylk, Itga1, Mgh11, Sorbs1, Col1a2, Lamb1, Itga7, Jam3, Lamb2, Itgb1, Bsg, Akr1c14, Ccl28, Atp8b1, Fignl1, Aspa, Gramd3, Trim59, Gm35315, Lpar4, Calcrl, Prrx1, LOC118567992, Zfp979, Ddias, 4930447C04Rik, Zfp938, Ndufb1, Nox1, Hacd4, Slc7a11, Cntf, Cdc42ep2, Sp7, Clec2d, Zfp977, Gadl1, Zfp469, Ada, Hif3a, Zbtb16, Hr, Tekt4, Hspa1b, C2cd4a, Eps812, Ppp1r1b, Fbxw23, Cd101, Ripk4, Bdkrb2, Grin2c, Vgll2, Cacng8, Gm20346, Sf3a2, Egr4, Hspa1a, Rsph1, Lamb3, Ppp1ccb, Dynlt1b, Sdhaf4, 1110025M09Rik, 9430078G10Rik, Hmgb3, Bex3, Tmsb10, Myct1, Ccdc116, Dbp, Apcdd1, Rnf122, Map4k1, Mypn, Il17rd, Rtbdn, Gjb2, Slc6a20a, Alx3, Foxd1, Tnxb, Ntsr1, Rab7b, Edn3, Wdr62, Ppp1r3g, Irf3, Klf14, Cryab, Hsph1, Hsp90b1, Hspa5, Hspe1, Plekhg4, Galm, Pcsk1, Rps19bp1, Mt3, Rp135a, Paqr5, Smim3, Cbln4, Adi1, Anln, Sgpp2, Tm2d3, Rps23rg1, Plagl1, Rtl8b, Sptssb, Vmn2497, adam6b, Dmrta1, Dbx2, LOC118568475, P2ryl0b, Pgap1, Slc9a7, Zbed6, Gm2808, Gm614, Npas4, St18, Gas213, Ankdd1a, Olfr111, Atp6v0d2, Gpnmb, Gm20056, Tm4sf19, 117r, Ms4a14, Otop1, Klra10, Ryr2, Baiap212, Gkn3, Rimklb, Tccal3, Aqp5, Ttc9, Cypla1, 2810459M11Rik, Grem2, Slitrk5, Sox 10, Acox2, Rsph1, Gm42517, Atp5k, Hbb-bt, Hba-a1, Hbb-bs, Slc2a5, Slc25a1, H2-Q10, Tafa5, Tbx1, Chchd10, Cox8b, Cnksr1, Gck, Aqp8, Hspb1, Hsp90aa1, Wnt4, Col11a2, Cd16311, Neurl1a, Map3k7cl, Tpm2, My19, Tagln, Eva1c, Gmfg, Rap1gap, Sncg, Mylk, Slc8a2, Eps812, Prag1, Capn3, Tmem 150c, Kcnc1, Vipr1, 2610044015Rik8, AW551984, Zfp442, Klhl32, Tfrc, Tmem35a, Kcne1, Mlf1, Tmx1, Pirt, Smim3, Fgf9, Vegfc, Tet1, Lrch2, Car8, Klhl4, Gm6712, Meox2, Ifitm1, Lepr, Gm11100, Scgb1c1, Gins1, Lhx6, Gm52351, Gdf15, Chac1, Vstm21, Ms4a2, Cma2, Mcpt9, Fccr1a, Mcpt4, Cpa3, Mcpt1, Mcpt2, Calca, Pbp2, Misp3, Cnbd2, Smin22, H4c9, Atp5k, Hypk, Ndufa2, Timm13, Uqcr11, H4c8, Tmsb10, Abca8a, Acvr1c, Gm39469, Scai, Gucy1a2, Irs1, Frem2, Cntin, Gm10033, Cd36, Scd1, Pi15, Zfp979, Gm8369, Ednrb, Tnfsf10, Hhip, Fam126b, Lrrc19, Phlpp2, Chrm2, Kctd12b, 1118, Cyp2c68, Hmcn1, Tnfrsf12a, Cma1, Mcpt4, Dact2, Itgb7, Banp, Nos1ap, Ppp1r14b1, Catsper4, Bmp8a, Phlda1, Slc25a30, Kif26b, Tead4, Chia1, Pifo, Tekt1, Kcnab1, Otub2, Hip1r, Syt9, Pkp2, Shc2, Jchain, Pard6b, Ptx3, Sfxn2, Socs2, Abhd18, Arhgef26, Peg3, Pfkfb3, Fsbp, Mc5r, Conf, Chrdl2, Zfp503, 4930563E22Rik, Sqle, Erfe, Cdc14a, Slc7a2, Trp53i11, Bcl6b, Ptger4, Ctla2a, Tspan6, Gpx8, Elovl3, Fgfbp1.

### Embodiment 16

The method of embodiment 14, wherein the at least one circulating metabolite is 3-indole carboxylic acid glucuronid, 6-Hydroxy-5-methoxyindole glucuronide, Hmba (2e,4e,6e,8e,10e,12e,14e,16e,18e,20e,22e)-2,4,6,8,10,12,14,16,18,20,22-tetracosaundecaenal, S-3-oxodecanoyl cysteamine, (2e,4e,8z)-n-(2-hydroxy-2-methylpropyl)-2,4,8-tetradecatrienamide, 4-Oxo-1-(3-pyridyl)-1-butanone, Methyprylon, 3,4-dimethylbenzoic acid, Asarone, Brassicanal a, 4-ethyl-2,6-dihydroxyphenyl hydrogen sulfate, Andrographolide, N-acetyl-l-tyrosine, Indole-3-carbidol, Valerophenone, 4-tert-amylphenol, Leu-val, 3-[2-(hydroxymethyl)-4-methoxyphenyl]-6-methoxy-4-oxo-3,4-dihydro-1 (2h)-quinazolinecarbaldehyde, Olodaterol, Esmolol, 5-hydroxyindoleacetate, or Indole-3-carboxilic acid-o-sulphate.

### REFERENCES

Athauda, D., Maclagan, K., Skene, S. S., Bajwa-Joseph, M., Letchford, D., Chowdhury, K., Hibbert, S., Budnik, N., Zampedri, L., Dickson, J., Li, Y., Aviles-Olmos, I., Warner, T. T., Limousin, P., Lees, A. J., Greig, N. H., Tebbs, S., & Foltynie, T. (2017). Exenatide once weekly versus placebo in Parkinson's disease: a randomised, double-blind, placebo-controlled trial. The Lancet, 390(10103), 1664-1675. see worldwide website: doi.org/10.1016/S0140-6736 (17) 31585-4
Aviles-Olmos, I., Dickson, J., Kefalopoulou, Z., Djamshidian, A., Kahan, J., Ell, P., Whitton, P., Wyse, R., Isaacs, T., Lees, A., Limousin, P., & Foltynie, T. (2014). Motor and cognitive advantages persist 12 months after exenatide exposure in Parkinson's disease. Journal of Parkinson's Disease, 4(3), 337-344. see worldwide website: doi.org/10.3233/JPD-140364
Bertilsson, G., Patrone, C., Zachrisson, O., Andersson, A., Dannaeus, K., Heidrich, J., Kortesmaa, J., Mercer, A., Nielsen, E., Rönnholm, H., & Wikström, L. (2008). Peptide hormone exendin-4 stimulates subventricular zone neurogenesis in the adult rodent brain and induces recovery in an animal model of Parkinson's disease. Journal of Neuroscience Research, 86 (2), 326-338. see worldwide website: doi.org/10.1002/JNR.21483
Cohen, A. A., Ferrucci, L., Fülöp, T., Gravel, D., Hao, N., Kriete, A., Levine, M. E., Lipsitz, L. A., Olde Rikkert, M. G. M., Rutenberg, A., Stroustrup, N., & Varadhan, R. (2022). A complex systems approach to aging biology. Nature Aging 2022 2:7, 2(7), 580-591. see worldwide website: doi.org/10.1038/s43587-022-00252-6
Fan, Y., Yi, Z., D'Agati, V. D., Sun, Z., Zhong, F., Zhang, W., Wen, J., Zhou, T., Li, Z., He, L., Zhang, Q., Lee, K., He, J. C., & Wang, N. (2019). Comparison of kidney transcriptomic profiles of early and advanced diabetic nephropathy reveals potential new mechanisms for disease progression. Diabetes, 68(12), 2301-2314. see worldwide website: doi.org/10.2337/DB19-0204/-/DC1
Heuvelman, V. D., Van Raalte, D. H., & Smits, M. M. (2020). Cardiovascular effects of glucagon-like peptide 1 receptor agonists: from mechanistic studies in humans to clinical outcomes. Cardiovascular Research, 116(5), 916-930. see worldwide website: doi.org/10.1093/CVR/CVZ323
Hinnen, D. (2017). Glucagon-like peptide 1 receptor agonists for type 2 diabetes. Diabetes Spectrum, 30(3), 202-210. see worldwide website: doi.org/10.2337/ds16-0026
Kaeberlein, M., Powers, R. W., Steffen, K. K., Westman, E. A., Hu, D., Dang, N., Kerr, E. O., Kirkland, K. T., Fields, S., & Kennedy, B. K. (2005). Regulation of yeast replicative life span by TOR and Sch9 in response to nutrients. Science (New York, N.Y.), 310(5751), 1193-1196. see worldwide website: doi.org/10.1126/SCIENCE.1115535
Kreiner, F. F., Hovingh, G. K. K., & von Scholten, B. J. (2022). The potential of glucagon-like peptide-1 receptor agonists in heart failure. Frontiers in Physiology, 13, 983961. see worldwide website: doi.org/10.3389/FPHYS.2022.983961/BIBTEX
Li, Y., Duffy, K. B., Ottinger, M. A., Ray, B., Bailey, J. A., Holloway, H. W., Tweedie, D., Perry, T., Mattson, M. P., Kapogiannis, D., Sambamurti, K., Lahiri, D. K., & Greig, N. H. (2010). GLP-1 receptor stimulation reduces amyloid-beta peptide accumulation and cytotoxicity in cellular and animal models of Alzheimer's disease. Journal of Alzheimer's Disease: JAD, 19(4), 1205-1219. see worldwide website: doi.org/10.3233/JAD-2010-1314
Li, Y., Perry, T. A., Kindy, M. S., Harvey, B. K., Tweedie, D., Holloway, H. W., Powers, K., Shen, H., Egan, J. M., Sambamurti, K., Brossi, A., Lahiri, D. K., Mattson, M. P., Hoffer, B. J., Wang, Y., & Greig, N. H. (2009). GLP-1 receptor stimulation preserves primary cortical and dopaminergic neurons in cellular and rodent models of stroke and Parkinsonism. Proceedings of the National Academy of Sciences of the United States of America, 106(4), 1285-1290. see worldwide website: doi.org/10.1073/PNAS.0806720106
Li, Z., Chen, X., Vong, J. S. L., Zhao, L., Huang, J., Yan, L. Y. C., Ip, B., Wing, Y. K., Lai, H. M., Mok, V. C. T., & Ko, H. (2021). Systemic GLP-1R agonist treatment reverses mouse glial and neurovascular cell transcriptomic aging signatures in a genome-wide manner. Communications Biology 2021 4:1, 4(1), 1-6. see worldwide website: doi.org/10.1038/s42003-021-02208-9
López-Otín, C., Blasco, M. A., Partridge, L., Serrano, M., & Kroemer, G. (2023). Hallmarks of aging: An expanding universe. Cell, 186(2), 243-278. see worldwide website: doi.org/10.1016/J.CELL.2022.11.001
Mannick, J. B., & Lamming, D. W. (2023). Targeting the biology of aging with mTOR inhibitors. Nature Aging 2023 3:6, 3(6), 642-660. see worldwide website: doi.org/10.1038/s43587-023-00416-y
McLean, B. A., Wong, C. K., Campbell, J. E., Hodson, D. J., Trapp, S., & Drucker, D. J. (2021). Revisiting the Complexity of GLP-1 Action from Sites of Synthesis to Receptor Activation. Endocrine Reviews, 42(2), 101-132. see worldwide website: doi.org/10.1210/ENDREV/BNAA032
Miller, M. E., Byington, R. P., Goff, vid C., Forest, W., York, N., Buse, J. B., Genuth, S., Ismail, F., & Grimm, R. H. (2008). Effects of Intensive Glucose Lowering in Type 2 Diabetes. New England Journal of Medicine, 358(24), 2545-2559. see worldwide website: doi.org/10.1056/NEJMOA0802743/SUPPL_FILE/NEJM_ACCORD_2545SA1.PDF
Mima, A., Hiraoka-Yamomoto, J., Li, Q., Kitada, M., Li, C., Geraldes, P., Matsumoto, M., Mizutani, K., Park, K., Cahill, C., Nishikawa, S. I., Rask-Madsen, C., & King, G. L. (2012). Protective effects of GLP-1 on glomerular endothelium and its inhibition by PKCB activation in diabetes. Diabetes, 61(11), 2967-2979. see worldwide website: doi.org/10.2337/DB11-1824
Perry, T. A., Lahiri, D. K., Sambamurti, K., Chen, D., Mattson, M. P., Egan, J. M., & Greig, N. H. (2003). Glucagon-like peptide-1 decreases endogenous amyloid-beta peptide (Abeta) levels and protects hippocampal neurons from death induced by Abeta and iron. Journal of Neuroscience Research, 72(5), 603-612. see worldwide website: doi.org/10.1002/JNR.10611
Vellai, T., Takacs-Vellai, K., Zhang, Y., Kovacs, A. L., Orosz, L., & Müller, F. (2003). Influence of TOR kinase on lifespan in C. elegans. Nature 2003 426:6967, 426(6967), 620-620. see worldwide website: doi.org/10.1038/426620a
Zhao, L., Li, Z., Vong, J. S. L., Chen, X., Lai, H. M., Yan, L. Y. C., Huang, J., Sy, S. K. H., Tian, X., Huang, Y., Chan, H. Y. E., So, H. C., Ng, W. L., Tang, Y., Lin, W. J., Mok, V. C. T., & Ko, H. (2020). Pharmacologically reversible zonation-dependent endothelial cell transcriptomic changes with neurodegenerative disease associations in the aged brain. Nature Communications, 11(1), 1-15. see worldwide website: doi.org/10.1038/s41467-020-18249-3

We claim:

1. A method for decreasing an aging-associated functional change in a subject, the method comprising administering to the subject an effective amount of a glucagon-like peptide 1 receptor agonist (GLP-1RA); wherein the GLP-1RA is selected from the group consisting of exenatide, liraglutide, lixisenatide, albiglutide, dulaglutide, semaglutide, taspoglutide, PF-06882961, OWL-833, TTP-273, LY3502970, a derivative of exenatide, a derivative of liraglutide, a derivative of lixisenatide, a derivative of albiglutide, a derivative of dulaglutide, a derivative of semaglutide, a derivative of taspoglutide, a derivative of PF-06882961, a derivative of OWL-833, a derivative of TTP-273, and a derivative of LY3502970.

2. The method of claim 1, wherein the subject is a mammal.

3. The method of claim 2, wherein the mammal is a primate.

4. The method of claim 3, wherein the primate is a human.

5. The method of claim 1, wherein the subject is 50 years or older.

6. The method of claim 1, wherein the aging-associated functional change is a reduction of muscle strength, balance, muscle endurance, or any combination thereof.

7. The method of claim 1, wherein the aging-associated functional change is a decrease in heart ejection fraction.

8. The method of claim 1, wherein the aging-associated function impairment is cognitive impairment.

9. The method of claim 1, wherein the aging-associated functional change is a measurable change in colon activity, adipose tissue amount, circulating white blood cell amount, spleen activity, lung airflow, skeletal muscle activity, heart activity, liver activity, kidney activity, or any combination thereof.

10. The method according to claim 9, wherein the measurable change is measured by a functional imaging or recording method.

11. The method according to claim 10, wherein the functional imaging or recording method is spirometry, echocardiogram, functional magnetic resonance imaging (fMRI), magnetic resonance imaging (MRI), hyperpolarized carbon-13 (¹³C) magnetic resonance spectroscopic imaging (MRSI), ultrasonography (US), positron emission tomography (PET), single-photon emission computerized tomography (SPECT), electroencephalography (EEG), magnetoencephalography (MEG), functional near-infrared spectroscopy (fNIRS), intracortical electrode recording, or deep brain electrode recording.

12. The method of claim 1, wherein the derivative of exenatide is an exenatide-antibody conjugate, an exenatide-peptide conjugate, an exenatide-nucleotide conjugate, or an exenatide-polyethylene glycol conjugate;
the derivative of liraglutide is a liraglutide-antibody conjugate, a liraglutide-peptide conjugate, a liraglutide-nucleotide conjugate, or a liraglutide-polyethylene glycol conjugate;
the derivative of lixisenatide is a lixisenatide-antibody conjugate, a lixisenatide-peptide conjugate, a lixisenatide-nucleotide conjugate, or a lixisenatide-polyethylene glycol conjugate;
the derivative of albiglutide is an albiglutide-antibody conjugate, an albiglutide-peptide conjugate, an albiglutide-nucleotide conjugate, or an albiglutide-polyethylene glycol conjugate;
the derivative of dulaglutide is a dulaglutide-antibody conjugate, a dulaglutide-peptide conjugate, a dulaglutide-nucleotide conjugate, or a dulaglutide-polyethylene glycol conjugate;
the derivative of semaglutide is a semaglutide-antibody conjugate, a semaglutide-peptide conjugate, a semaglutide-nucleotide conjugate, or a semaglutide-polyethylene glycol conjugate;
the derivative of taspoglutide is a taspoglutide-antibody conjugate, a taspoglutide-peptide conjugate, a taspoglutide-nucleotide conjugate, or a taspoglutide-polyethylene glycol conjugate;
the derivative of PF-06882961 is a PF-06882961-antibody conjugate, a PF-06882961-peptide conjugate, a PF-06882961-nucleotide conjugate, or a PF-06882961-polyethylene glycol conjugate;
the derivative of OWL-833 is an OWL-833-antibody conjugate, an OWL-833-peptide conjugate, an OWL-833-nucleotide conjugate, or an OWL-833-polyethylene glycol conjugate;
the derivative of TTP-273 is a TTP-273-antibody conjugate, a TTP-273-peptide conjugate, a TTP-273-nucleotide conjugate, or a TTP-273-polyethylene glycol conjugate; or
the derivative of LY3502970 is a LY3502970-antibody conjugate, a LY3502970-peptide conjugate, a LY3502970-nucleotide conjugate, or a LY3502970-polyethylene glycol conjugate.

13. The method of claim 1, wherein the aging-associated functional change is a change in an expression of at least one gene in the subject.

14. The method of claim 1, wherein the aging-associated functional change is a change in a level of at least one circulating metabolite in the subject.

15. The method of claim 13, wherein the at least one gene is C1qa, C1qb, C1qc, C4b, B2m, Tap2, H2-D1, H2-K1, Sparcl1, Gpc6, Tgfb2, Megf10, Mertk, Chrdl1, Kcnj10, Kcnn2, Slc1a2, Slc1a3, Slc6a1, Slc6a9, Slc6a11, Slc7a10, Slc7a11, Slc16a1, Srebf1, Gja1, Gjb6, Itpr2, Grm3, Gria2, Gabbr1, Gabbr2, Csf2r, P2ryl3, Appe, Ccl3, Ccl4, Cd52, Cst7, Fabp5, Tyrobp, Cd14, Cd33, Ifngr1, Ly86, Map4k4, Cd300a, Il1Ora, Il10rb, Camk2g, Stim1, Gsn, Atp2a2, Inpp4b, Mcur1, S100a6, Tspo, Mylk, Itga1, Mgh11, Sorbs1, Col1a2, Lamb1, Itga7, Jam3, Lamb2, Itgb1, Bsg, Akr1c14, Ccl28, Atp8b1, Fignl1, Aspa, Gramd3, Trim59, Gm35315, Lpar4, Calcrl, Prrx1, LOC118567992, Zfp979, Ddias, 4930447C04Rik, Zfp938, Ndufb1, Nox1, Hacd4, Slc7a11, Cntf, Cdc42ep2, Sp7, Clec2d, Zfp977, Gadl1, Zfp469, Ada, Hif3a, Zbtb16, Hr, Tekt4, Hspa1b, C2cd4a, Eps812, Ppp1r1b, Fbxw23, Cd101, Ripk4, Bdkrb2, Grin2c, Vgl12, Cacng8, Gm20346, Sf3a2, Egr4, Hspa1a, Rsph1, Lamb3, Ppp1ccb, Dynlt1b, Sdhaf4, 1110025M09Rik, 9430078G10Rik, Hmgb3, Bex3, Tmsb10, Myct1, Ccdc116, Dbp, Apcdd1, Rnf122, Map4k1, Mypn, Il17rd, Rtbdn, Gjb2, Slc6a20a, Alx3, Foxd1, Tnxb, Ntsr1, Rab7b, Edn3, Wdr62, Ppp1r3g, Irf3, Klf14, Cryab, Hsph1, Hsp90b1, Hspa5, Hspe1, Plekhg4, Galm, Pcsk1, Rps19bp1, Mt3, Rp135a, Paqr5, Smim3, Cbln4, Adi1, Anln, Sgpp2, Tm2d3, Rps23rg1, Plagl1, Rtl8b, Sptssb, Vmn2497, adam6b, Dmrta1, Dbx2, LOC118568475, P2ryl0b, Pgap1, Slc9a7, Zbed6, Gm2808, Gm614, Npas4, St18, Gas213, Ankdd1a, Olfr111, Atp6v0d2, Gpnmb, Gm20056, Tm4sf19, Il7r, Ms4a14, Otop1, Klra10, Ryr2, Baiap212, Gkn3, Rimklb, Tceal3, Aqp5, Ttc9, Cypla1, 2810459M11Rik, Grem2, Slitrk5, Sox10, Acox2, Rsph1, Gm42517, Atp5k, Hbb-bt, Hba-a1, Hbb-bs, Slc2a5, Slc25a1, H2-Q10, Tafa5, Tbx1, Chchd10, Cox8b, Cnksr1, Gck, Aqp8, Hspb1, Hsp90aa1, Wnt4, Col11a2, Cd16311, Neurl1a, Map3k7cl, Tpm2, My19, Tagln, Eva1c, Gmfg, Rap1gap, Sncg, Mylk, Slc8a2, Eps812, Prag1, Capn3, Tmem150c, Kcnc1, Vipr1, 2610044015Rik8, AW551984, Zfp442, Klh132, Tfrc, Tmem35a, Kcne1, Mlf1, Tmx1, Pirt, Smim3, Fgf9, Vegfc, Tet1, Lrch2, Car8, Klh14, Gm6712, Meox2, Ifitm1, Lepr, Gm11100, Scgb1c1, Gins1, Lhx6, Gm52351, Gdf15, Chac1, Vstm21, Ms4a2, Cma2, Mcpt9, Fcer1a, Mcpt4, Cpa3, Mcpt1, Mcpt2, Calca, Pbp2, Misp3, Cnbd2, Smin22, H4c9, Atp5k, Hypk, Ndufa2, Timm13, Uqcr11, H4c8, Tmsb10, Abca8a, Acvr1c, Gm39469, Scai, Gucy1a2, Irs1, Frem2, Cntin, Gm10033, Cd36, Scd1, Pi15, Zfp979, Gm8369, Ednrb, Tnfsf10, Hhip, Fam126b, Lrrc19, Phlpp2, Chrm2, Kctd12b, 1118, Cyp2c68, Hmen1, Tnfrsf12a, Cma1, Mcpt4, Dact2, Itgb7, Banp, Nos1ap, Ppp1r14b1, Catsper4, Bmp8a, Phlda1, Slc25a30, Kif26b, Tead4, Chia1, Pifo, Tekt1, Kcnab1, Otub2, Hip1r, Syt9, Pkp2, Shc2, Jchain, Pard6b, Ptx3, Sfxn2, Socs2, Abhd18, Arhgef26, Peg3, Pfkfb3, Fsbp, Mc5r, Conf, Chrdl2, Zfp503, 4930563E22Rik, Sqle, Erfe, Cdc14a, Slc7a2, Trp53i11, Bcl6b, Ptger4, Ctla2a, Tspan6, Gpx8, Elovl3, or Fgfbp1.

16. The method of claim 14, wherein the at least one circulating metabolite is 3-indole carboxylic acid glucuronid, 6-Hydroxy-5-methoxyindole glucuronide, Hmba, (2e,4e,6e,8e, 10e, 12e, 14e, 16e, 18e,20e,22e)-2,4,6,8,10,12, 14,16, 18,20,22-tetracosaundecaenal, S-3-oxodecanoyl cysteamine, (2e,4e,8z)-n-(2-hydroxy-2-methylpropyl)-2,4,8-tetradecatrienamide, 4-Oxo-1-(3-pyridyl)-1-butanone, Methyprylon, 3,4-dimethylbenzoic acid, Asarone, Brassicanal a, 4-ethyl-2,6-dihydroxyphenyl hydrogen sulfate, Andrographolide, N-acetyl-l-tyrosine, Indole-3-carbidol, Valerophenone, 4-tert-amylphenol, Leu-val, 3-[2-(hydroxymethyl)-4-methoxyphenyl]-6-methoxy-4-oxo-3,4-dihydro-1 (2h)-quinazolinecarbaldehyde, Olodaterol, Esmolol, 5-hydroxyindoleacetate, or Indole-3-carboxilic acid-o-sulphate.


SEQUENCE LISTING (6 sequences)

SEQ ID NO: 1 - RNA, 21 nt, synthetic construct
  source 1..21: mol_type = other RNA
gcgtcaactttcttatcttca

SEQ ID NO: 2 - DNA, 21 bp, synthetic construct
  source 1..21: mol_type = other DNA
cctaaggttaagtcgccctcg

SEQ ID NO: 3 - DNA, 20 bp, synthetic construct
  source 1..20: mol_type = other DNA
cagtggggtacgcactttct

SEQ ID NO: 4 - DNA, 20 bp, synthetic construct
  source 1..20: mol_type = other DNA
taacgaacagcagcggaact

SEQ ID NO: 5 - DNA, 20 bp, synthetic construct
  source 1..20: mol_type = other DNA
ggcggagatgatgacccttt

SEQ ID NO: 6 - DNA, 20 bp, synthetic construct
  source 1..20: mol_type = other DNA
catcttccaggagcgagacc